(19) United States
(12) Patent Application Publication
(10) Pub. No.: US 2025/0147012 A1
(43) Pub. Date: May 8, 2025

(54) METHODS AND MICROFLUIDIC DEVICES FOR CHIROPTICAL DETECTION AND MUTATION ANALYSIS OF CANCER-ASSOCIATED EXTRACELLULAR VESICLES

(71) Applicant: Yoon-Tae KANG

(72) Inventor: Yoon-Tae KANG

(21) Appl. No.: 18/579,459

(22) PCT Filed: Jul. 15, 2022
(86) PCT No.: PCT/US2022/037283

§ 371 Date: Jan. 15, 2024

(51) Int. Cl. G01N 33/543; B01L 3/00; G01N 33/574

(52) U.S. Cl. CPC G01N 33/54366; B01L 3/502707; B01L 3/502761; G01N 33/54346; G01N 33/57423; B01L 2200/0647; B01L 2200/12; B01L 2300/12; B01L 2300/168

(57) ABSTRACT

Microfluidic devices including a microfluidic channel with at least one surface having a plurality of chiral nanoparticles disposed thereon include a light-absorbing material (e.g., gold) and a targeting ligand capable of binding to a bioactive target analyte in a biological fluid sample. The bioactive target analyte can indicate presence of cancerous cells or mutated proteins in the biological fluid sample taken from a subject. Chiral gold nanoparticle layer-by-layer assembled onto a microfluidic device can rapidly isolate and profile cancer-associated exosomes directly from blood plasma using their own unique chiral signal. Exosomes from lung cancer patients can be distinguished from healthy donor exosomes by chiroptical spectroscopic signatures of biomolecular components. Mutation/deletion of epidermal growth factor receptor are also characterized, suggesting the possibility for in-depth mutation profiling in addition to cancer diagnostics. Methods of detecting bioactive target analytes and making the microfluidic devices are also provided.

### CROSS-REFERENCE TO RELATED APPLICATIONS

This application claims the benefit of U.S. Provisional Application No. 63/222,190, filed on Jul. 15, 2021. The entire disclosure of the above application is incorporated herein by reference.

### GOVERNMENT SUPPORT

This invention was made with government support under 1463474 and 1566460 awarded by the National Science Foundation, under CA202867 and CA208335 awarded by the National Institutes of Health, and under N00014-18-1-2876 awarded by the U.S. Office of Naval Research. The Government has certain rights in the invention.

### FIELD

The present disclosure relates to chiroptical detection and mutation analysis of cancer-associated extracellular vesicles in microfluidic devices, including methods of using and making such microfluidic devices.

### BACKGROUND

This section provides background information related to the present disclosure which is not necessarily prior art.

Most cells secrete extracellular vesicles (EVs) of nanoscale dimensions that carry informative cargo-containing proteins, lipids, and nucleic acids. It appears EVs play essential roles in cell-cell communication. Furthermore, the cargo and membrane proteins of small EVs, such as cancer-derived exosomes, reflect biological activity and status of malignant cells they are secreted by, playing an important role in cancer progression and metastatic destination. Recently, it has been shown that exosomes may carry the mutated proteins reflective of their cellular origin, prompting ongoing studies of these exosomes as prominent biomarkers for cancer diagnosis. Thus, cancer-cell secreted nanoscale small extracellular vesicles (sEVs); known as exosomes, represent a rapidly emerging family of biomarkers for cancer detection. While being high in information content, the current protocols for profiling sEVs require complex procedures and equipment involving exosome purification, which prevents their utilization in timely diagnosis of malignancies. For example, conventional protein profiling methods, such as western blot and enzyme linked immunosorbent assays (ELISA) rely on the use of monoclonal antibodies, require large amounts of proteins, and involve multi-step purification processes, which represent the central technological threshold for analysis of exosomes and other extracellular vesicles as liquid biopsies.

Microfluidic technologies have been used for sensing of different types of exosomes, but in limited capacity and with varying degrees of success. Recent advances in optical components for microfluidic systems led to improvements in their detection limit and information content. For example, surface-enhanced Raman scattering (SERS) offer signal amplification and real-time detection capabilities as exemplified by detection of immune checkpoint molecules and cancer exosomes. Relatively low intensity and non-linearity of SERS necessitates; however, considerable sample pre-processing and data post-processing, which makes the process lengthy and imposes limitations on its use in clinical settings. Concurrently, a microfluidic platform for exosome capture can also be utilized for surface plasmon resonance (SPR) assay using periodic nanohole arrays. These substrates require; however, sophisticated fabrication process and are difficult to implement in inexpensive polymer/glass-based devices due to optical limitations of SPR. While studies on both SERS and SPR represent progress in quantification of specific antigen presentation on exosomes, it is challenging to use these technologies for detection of unique proteins on exosomes without specific antibodies. It would be advantageous to develop accurate, rapid, streamlined methods of detecting exosomes or other mutations in proteins directly from blood plasma in improved microfluidic devices.

### SUMMARY

This section provides a general summary of the disclosure, and is not a comprehensive disclosure of its full scope or all of its features.

In certain aspects, the present disclosure relates to a microfluidic device comprising a microfluidic channel comprising at least one surface having a plurality of chiral nanoparticles disposed thereon. The plurality of chiral nanoparticles each comprise a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof. The plurality of chiral nanoparticles also each comprise a targeting ligand associated with the plurality of chiral nanoparticles that is capable of binding to a bioactive target analyte in a biological fluid sample. The bioactive target analyte indicates a presence of cancerous cells or mutated proteins in the biological fluid sample.

In one aspect, the microfluidic channel comprises a multilayered coating formed by a layer-by-layer deposition process that comprises a plurality of positive layers interspersed with a plurality of negative layers. An exposed surface of the multilayered coating comprises the plurality of chiral nanoparticles having a positive charge.

In one further aspect, at least one layer of the plurality of positive layers comprises a cationic poly(dimethyldiallylammonium chloride) (PDDA) and at least one layer of the plurality of negative layers comprises an anionic polystyrene sulfonate (PSS).

In one aspect, the at least one surface of the microfluidic channel is plasma etched.

In one aspect, the microfluidic channel is formed on a microchip.

In one aspect, the bioactive target analyte is selected from the group consisting of: phosphatidylserine (PS), tetraspanin proteins, epithelial cancer adhesion molecule (EpCAM), epidermal growth factor receptor (EGFR), and combinations thereof.

In one aspect, the bioactive target analyte comprises phosphatidylserine (PS).

In one aspect, the plurality of chiral nanoparticles comprise chiral gold nanoparticles functionalized with mercaptoundecanoic acid (MUA) reacted with 1-ethyl-3-(3-dimethylaminopropyl)carbodiimide (EDC) and N-hydroxysuccinimide (NHS).

In one aspect, the targeting ligand is selected from the group consisting of: Annexin V, anti-CD63, anti-CD81, anti-CD9, anti-CD56, anti-CD-133, anti-EpCAM, anti-EGFR, anti-vimentin, and combinations thereof.

In one aspect, the bioactive target analyte comprises phosphatidylserine (PS) and the targeting ligand comprises Annexin V

In one further aspect, the targeting ligand further comprises deglycosylated avidin associated with biotin that is associated with Annexin V.

In certain further aspects, the present disclosure relates to a method of detecting a target bioactive analyte in a biological fluid sample obtained from a subject. The method comprises passing a biological fluid sample through a microfluidic channel comprising at least one surface having a plurality of chiral nanoparticles disposed thereon and directing circularly polarized light at the microfluidic channel while the biological fluid sample is disposed therein to measure a first level of at least one of magnitude of circular dichroism or peak wavelength. The plurality of chiral nanoparticles each comprise a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof. The plurality of chiral nanoparticles also each comprise a targeting ligand that is capable of binding to a bioactive target analyte optionally present in the biological fluid sample. The method comprises comparing the first level of at least one of magnitude of circular dichroism or peak wavelength to a baseline level of at least one of magnitude of circular dichroism or peak wavelength in the microfluidic channel in the absence of the biological fluid sample. A difference between the first level and the baseline level indicates a presence of the bioactive target analyte that indicates a presence of cancerous cells or mutated proteins in the biological fluid sample.

In one aspect, the method further comprises measuring the baseline level of at least one of magnitude of circular dichroism or peak wavelength by directing circularly polarized light at the microfluidic channel in the absence of the biological fluid sample.

In one aspect, the first level is a peak wavelength measured in a range of greater than or equal to about 520 nm to less than or equal to about 1.4 micrometer (μm).

In one aspect, the first level is a peak magnitude of circular dichroism and the baseline level is a peak magnitude of circular dichroism and the comparing shows a difference in peak magnitudes of circular dichroism between the first level and the baseline level.

In certain other aspects, the present disclosure relates to a method for forming a microfluidic device for detecting a bioactive target analyte in a biological fluid sample obtained from a subject. The method may comprise applying a first charged material having a first polarity to at least one surface of a microfluidic channel on a substrate having a second polarity opposite to the first polarity. The method also comprises applying a second charged material having the second polarity over the first charged material in a layer-by-layer process on the at least one surface. The first charged material and the second charged material are distinct from one another and define a layered coating. The method further comprises applying a plurality of chiral nanoparticles over the layered coating, so that the plurality of chiral nanoparticles are exposed to the microfluidic channel. Each of the plurality of chiral nanoparticles comprises a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof. The method also comprises functionalizing the plurality of chiral nanoparticles and associating each nanoparticle of the plurality with a targeting ligand that is capable of binding to the bioactive target analyte in the biological fluid sample, wherein the bioactive target analyte indicates a presence of cancerous cells or mutated proteins in the biological fluid sample.

In one aspect, the first charged material comprises a cationic poly(dimethyldiallylammonium chloride) (PDDA) and the second charged material comprises an anionic polystyrene sulfonate (PSS) and the plurality of chiral nanoparticles are cationic and have the first polarity.

In one aspect, the plurality of chiral nanoparticles are stabilized with a shape-directing ligand comprising at least one of L-cysteine or D-cysteine.

In one aspect, the method further comprises plasma etching the at least one surface of the microfluidic channel.

In one aspect, the substrate is a microchip and the microfluidic channel is formed on the microchip.

In one aspect, the plurality of chiral nanoparticles comprise chiral gold nanoparticles and the functionalizing comprises reacting the chiral gold nanoparticles with mercaptoundecanoic acid (MUA) followed by reacting with 1-ethyl-3-(3-dimethylaminopropyl)carbodiimide (EDC) and then reacting with N-hydroxysuccinimide (NHS) to form a plurality of functionalized chiral gold nanoparticles.

In one aspect, the targeting ligand is selected from the group consisting of: Annexin V, anti-CD63, anti-CD81, anti-CD9, anti-CD56, anti-CD-133, anti-EpCAM, anti-EGFR, anti-vimentin, and combinations thereof.

In one aspect, the bioactive target analyte comprises phosphatidylserine (PS) and the targeting ligand comprises Annexin V reacted with the plurality of functionalized chiral gold nanoparticles.

In one aspect, the targeting ligand further comprises first associating deglycosylated avidin with the plurality of functionalized chiral gold nanoparticles, then associating biotin with the deglycosylated avidin, followed by associating the biotin with Annexin V capable of binding to the bioactive target analyte

In one aspect, the method further comprises forming the plurality of chiral nanoparticles by growing triangular nanoplate precursors by adding gold precursor, a reductant, and a shape-directing ligand, followed by encapsulating the plurality of chiral nanoparticles in positively charged bilayer micelles.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flowchart showing a method of detecting a target bioactive analyte in a biological fluid sample obtained from a subject according to various aspects of the present disclosure.

FIGS. 2A-2L show geometry and optical properties of chiral gold nanoparticles (AuNPs) prepared in accordance with certain aspects of the present disclosure for exosome detection. FIGS. 2A-2B show scanning electron microscope (SEM) images of AuNPs synthesized with (FIG. 2A) L-Cysteine (L-Cys) and (FIG. 2B) D-Cysteine (D-Cys) as surface ligands. Magnified images (inset) show the handedness for intermediate (upper) and final structure (lower) of the NPs. FIG. 2C shows UV-vis absorbance spectra of chiral AuNPs synthesized with D-cysteine (Au_D-Cys, black) and L-cysteine (Au_L-Cys, gray). FIG. 2D shows a 3D Electron tomogram of Au_L-Cys. FIG. 2E shows circular dichroism (CD) spectra of AuNPs synthesized with L- and D-cysteine in mirror relationship. FIG. 2F shows CD spectrum of chiral AuNPs deposited on glass substrates used for exosome sensing CDEXO chip. FIGS. 2G-2J show a computational model of enantiomer AuNPs for us in electromagnetic simulation. FIG. 2G is a top view of an AuNP synthesized with L-Cys. FIG. 2H is a side view of the AuNP of FIG. 2G. FIG. 2I is a top view of an AuNP synthesized with D-Cys. FIG. 2J is a side view of the AuNP of FIG. 2J. FIGS. 2K-2L show a difference in extinction cross section (Δσ) under left-handed and right-handed circularly polarized light (σLCP−σRCP) calculated from the model in FIG. 2G for random orientation (FIG. 2K) and fixed orientation (FIG. 2L) of NPs in dielectric media in water and air, respectively.

FIGS. 3A-3I show a circular dichroism-based exosome sensing (CDEXO) chip prepared in accordance with certain aspects of the present disclosure for profiling of cancer-associated exosomes. FIG. 3A shows an engineering design of the microfluidic chip and principles of NP-exosome binding in the CDEXO chip. FIG. 3B shows schematics of the working principle and changes in optical properties induced by binding of exosomes to the NP layer. Exosome binding and elution events are monitored as chiroptical spectral shifts (Δλ) and magnitude changes (ΔCD) by the CDEXO chip using circular dichroism spectrometry. FIG. 3C is a schematic showing both preparation of the microfluidic channel, layer-by-layer deposition assembly, and functionalization of gold nanoparticles to have targeting ligands for reacting with bioactive target analytes in accordance with certain aspects of the present disclosure. FIGS. 3C-3D show preparation of the nanoparticle (NP) layer by via layer-by-layer deposition in a sensing region of a substrate including the CDEXO chip glass surface having a negative charge, a layer of positively charged poly(dimethyldiallylammonium chloride) (PDDA) disposed on top of the CDEXO chip glass surface, a layer of negatively charged anionic polystyrene sulfonate (PSS) disposed on top of the PDDA layer, and a layer of positively charged chiral AuNPs is assembled on top of the layer of PSS. FIG. 3E is a schematic showing surface modification/functionalization of chiral AuNP-layer for exosome isolation using standard 1-ethyl-3-(3-dimethylaminopropyl)carbodiimide and N-hydroxysuccinimide chemistry. FIGS. 3F-3I show example CDEXO chips prepared in accordance with certain aspects of the present disclosure for profiling of cancer-associated exosomes.

FIGS. 4A-4L show detection of cancer-associated exosomes with a CDEXO chip prepared in accordance with certain aspects of the present disclosure. FIG. 4A includes FIGS. 4A(i) and 4A(ii) showing photographs of a CDEXO exosome detection microfluidic device prepared in accordance with certain aspects of the present disclosure (FIG. 4A(i)) that can be seated within an insert component of a CD spectrometry system (FIG. 4A(ii)). FIGS. 4B-4C show SEM images of a CDEXO chip prepared in accordance with certain aspects of the present disclosure before exosome binding (FIG. 4B) and after exosome binding (FIG. 4C). FIG. 4D shows western blot analysis of exosomes from three different cancer cells carrying different EGFR mutations. FIG. 4E shows SEM images of chiral AuNPs on a CDEXO chip; where the inset shows exosomes (arrows) captured on the particles. FIG. 4F shows characteristic CD spectra of each cell line derived exosome. FIG. 4G shows CD signal changes before and after exosome binding and Ca²⁺ based exosome chelating using EDTA. FIGS. 4H-4I show detection sensitivity of CDEXO chip in terms of peak shift (FIG. 4H) and percentage change of CD signal (FIG. 4I). The CDEXO chip detection limit was determined by titrating a known concentration of H3255 exosomes and measuring their CD peak shift and signal change. FIG. 4J shows exosome isolation performance of CDEXO chip using three different cancer cell-derived exosomes comparing to control device without exosome capturing molecule conjugation. FIG. 4K shows CD-peak shift in three different cell line derived exosomes using CDEXO chip. FIG. 4L shows a percentage of change in CD-peak magnitude after exosome binding comparing to baseline CD peak.

FIGS. 5A-5F show profiling of cancer exosomes from clinical samples using a CDEXO chip prepared in accordance with certain aspects of the present disclosure. FIG. 5A shows representative signals of a healthy donor and a lung cancer patient after exosome isolation. FIG. 5B shows an amount of CD-peak shift and CD peak magnitude change for each sample. FIG. 5C-5D show examples of CD peak change for a sample having EGFR exon 19 deletion (FIG. 5C) and a sample having both EGFR L858R and T790M mutation (FIG. 5D). FIG. 5E shows a change of CD peak magnitude for 11 lung cancer patients, three samples with EGFR Exon 19 deletion and eight samples without Exon 19 deletion. FIG. 5F shows a percentage of change in CD-valley magnitude after exosome isolation comparing to baseline CD peak for 11 lung cancer patients with/without point mutation.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

In various aspects, the present disclosure provides methods of detecting a bioactive target analyte in a biological fluid sample obtained from a subject by use of a microfluidic device that will be described herein. In certain aspects, a microfluidic device comprises at least one microfluidic channel comprising at least one surface having a plurality of chiral nanoparticles disposed thereon. The at least one microfluidic channel is configured to receive a biological fluid sample for analysis that may contain a bioactive target analyte. As will be described further below, the plurality of chiral nanoparticles each comprise a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof. In certain aspects, the plurality of chiral nanoparticles comprise gold. The plurality of chiral nanoparticles also have an associated targeting ligand that is capable of binding to a bioactive target analyte in a biological fluid sample. As will be described further herein, the bioactive target analyte indicates a presence of cancerous cells or mutated proteins in the biological fluid sample.

The biological fluid sample may include bodily fluids, such as blood, serum, plasma, saliva, cerebrospinal fluid, urine, and the like. In certain aspects, the biological fluid sample comprises blood or plasma. The bioactive target analyte may be a bioactive material such as a cellular component (having a size smaller than a cell), protein, or other biological materials derived therefrom (e.g., nucleic acids, carbohydrates, lipids, proteins, polypeptides, amino acids, hormones, prostaglandins), by way of non-limiting example. In certain preferred aspects, a bioactive target analyte may be an extracellular vesicle, for example, selected from the group consisting of an exosome, a microvesicle, an apoptotic body, and combinations thereof. Exosomes are extracellular vesicles secreted by cells that can mirror cellular information from cells of origin. In certain variations, the bioactive target analyte comprises phosphatidyl-serine (PS) is an anionic phospholipid maintained on the inner-leaflet of a cell membrane and may be externalized in malignant cells and its exosomes and/or formed during apoptosis. In certain other aspects, a bioactive material may comprise a protein or polypeptide, such as a mutated protein. Alternatively or additionally, the bioactive target analyte may comprise another type of cell-derived material, such as tumor/extracellular vesicle specific proteins. The bioactive target analyte may be tetraspanin proteins (e.g., CD81, CD9, CD63, CD133, and CD56) or any other cancer-associated proteins such as epithelial cancer adhesion molecule (EpCAM), epidermal growth factor receptor (EGFR), and the like. For these bioactive target analytes, other antibodies targeting these aforementioned bioactive target analytes, like tumor/extracellular vesicle specific proteins, can be used as the targeting ligands on the chiral nanoparticles.

In certain variations, the bioactive target analyte originates from a cell and is present in a biological fluid sample, like blood or plasma, obtained from a subject to be treated, from another subject, or from another species. Further, the bioactive target analyte may originate from a eukaryotic cell, e.g., from an animal, such as a mammal. By way of non-limiting example, the mammal may be a human, domesticated companion animal, such as a cat or dog, or livestock, such as a cow, horse, sheep, goat, and the like.

As noted above, cancer-cell secreted nanoscale small extracellular vesicles (sEVs), known as exosomes, are biomarkers for cancer detection. As will be described further herein, in certain variations, chiral gold nanoparticles may be layer-by-layer assembled onto a surface of a microfluidic channel of a microfluidic device, which can rapidly isolate and detect cancer associated exosomes directly from blood plasma using their affinity to Annexin V (by binding with phosphatidyl-serine (PS)). Exosomes from lung cancer patients can be distinguished from those from healthy donors by chiroptical spectroscopic, or polarization rotation, signatures of biomolecular components of exosomes enhanced by chiral plasmonic nanoparticles. Additionally, bioactive target analytes may include mutated proteins. For example, mutation/deletion of epidermal growth factor receptors are also characterized herein, such that the present methods and microfluidic devices can be used for in-depth mutation profiling, in addition to cancer diagnostics.

Chirality is the foundational property of all biomolecules, which describes the fact the molecules and their mirror images cannot be superimposed, which manifests as different absorption coefficients for left- and right-circularly polarized light. Chiroptical activity of biomolecules is typically measured by circular dichroism (CD) spectrometry whose utilization, along, perhaps SERS, SPR and other methods, is highly desirable for exosome analysis because of the potentially large amount of information about membrane and cargo of exosomes and other extracellular vesicles contained in polarization rotation spectra. Thus, circular dichroism (CD) is differential absorption of left and right circularly polarized light. Circular dichroism may be expressed CD=extinction of left-handed circularly polarized light (LCP)—extinction of right handed circularly polarized light (RCP), where cross-section extinction is a sum of cross-section absorption and cross-section scattering.

However, extraction of this information and even their selective detection is challenging because chiroptical activity of proteins, lipids, and other biomolecules are typically confined to UV part of the spectrum, making detection of specific proteins in complex biological media very difficult using CD spectra. The present technology further provides the ability to overcome these challenges by taking advantage of the chiral inorganic nanostructures that can both enhance and shift CD peaks associated with specific proteins.

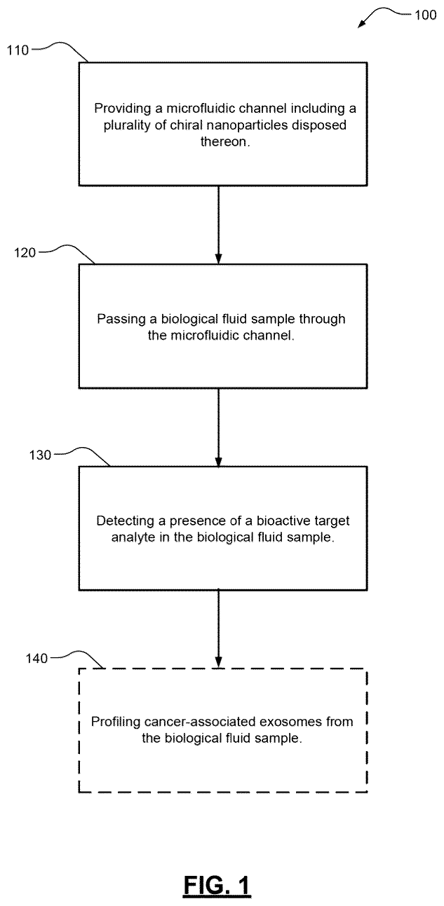

With reference to FIG. 1, the current technology provides a method 100 for detecting a bioactive target analyte in a biological fluid sample obtained from a subject. The method 100 generally includes providing a microfluidic channel including a plurality of chiral nanoparticles disposed thereon at 110. The microfluidic channel is configured to receive a biological fluid sample. In certain aspects, the method further comprises passing a biological fluid sample through the microfluidic channel at 120. A presence of a bioactive target analyte in the biological fluid sample may be detected at 130; and, if a bioactive target analyte is present, profiling the cancer-associated exosomes from the biological fluid sample at 140.

Optically convenient visible light range resonances of some chiral nanoparticles enable an attractive pathway for rapid and versatile profiling of various sEVs. High polarizability of self-assembled structures from gold nanoparticles (AuNPs) results in a strong plasmonic peak, which may correspond to red or near-infrared parts of the electromagnetic spectrum, which improves detection limits for many biomolecules and enables selective detection of fibrils and protein markers for various diseases. Generally, visible light has a wavelength visible ranging from about 390 to about 750 nm, with a red color having a wavelength in a range of about 625 nm to about 750 nm, while infrared radiation (IR) includes near infrared radiation (NIR) ranging from about 750 nm to about 1.4 micrometers (μm). In certain aspects, the emitted light is measured at greater than or equal to about 520 nm to less than or equal to about 1.4 μm, optionally from greater than or equal to about 520 nm to less than or equal to about 750 nm, and in certain variations, optionally from greater than or equal to about 625 nm to less than or equal to about 750 nm.

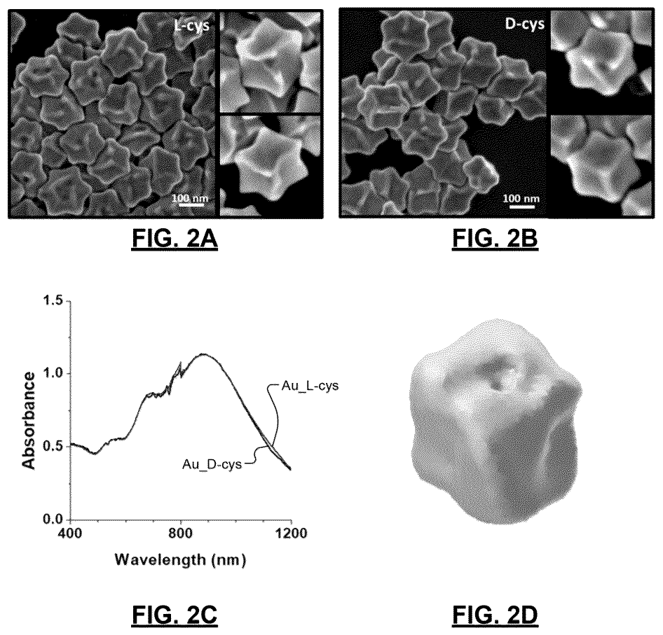

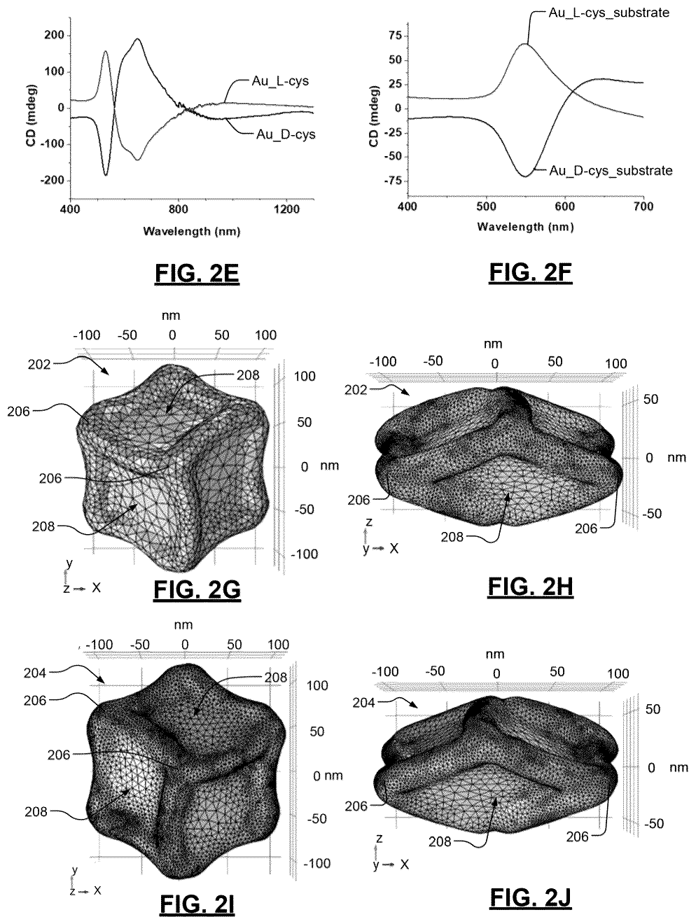

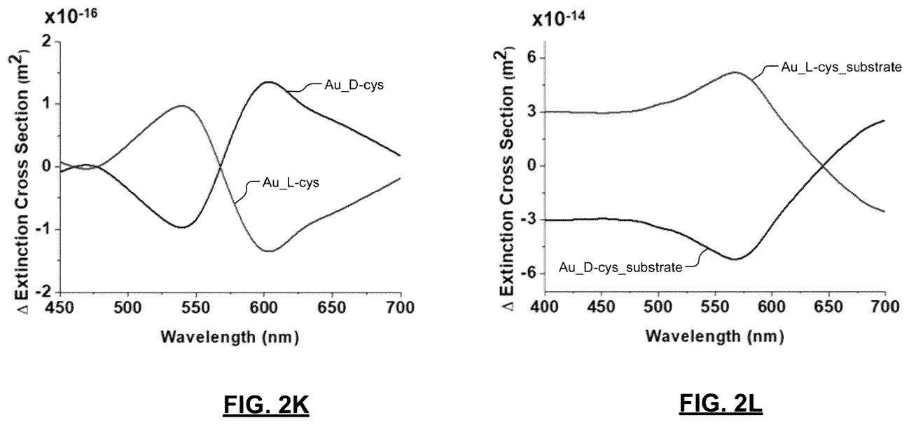

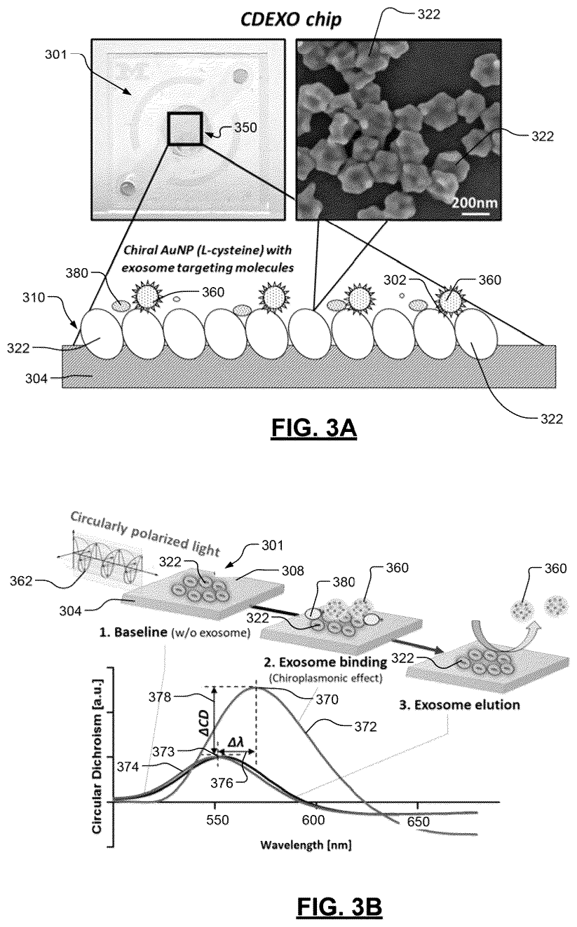

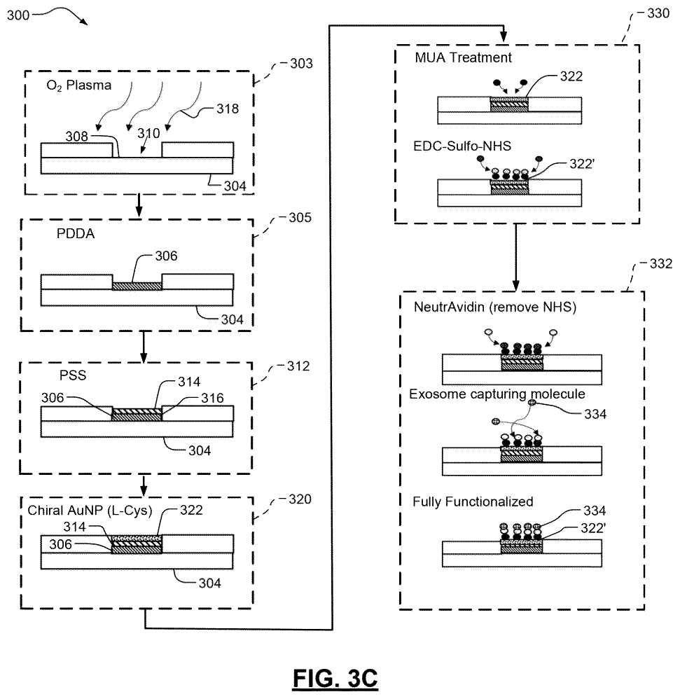

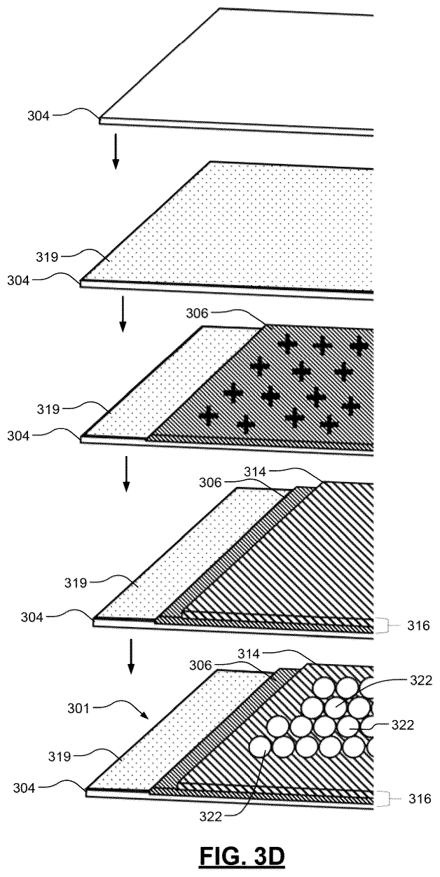

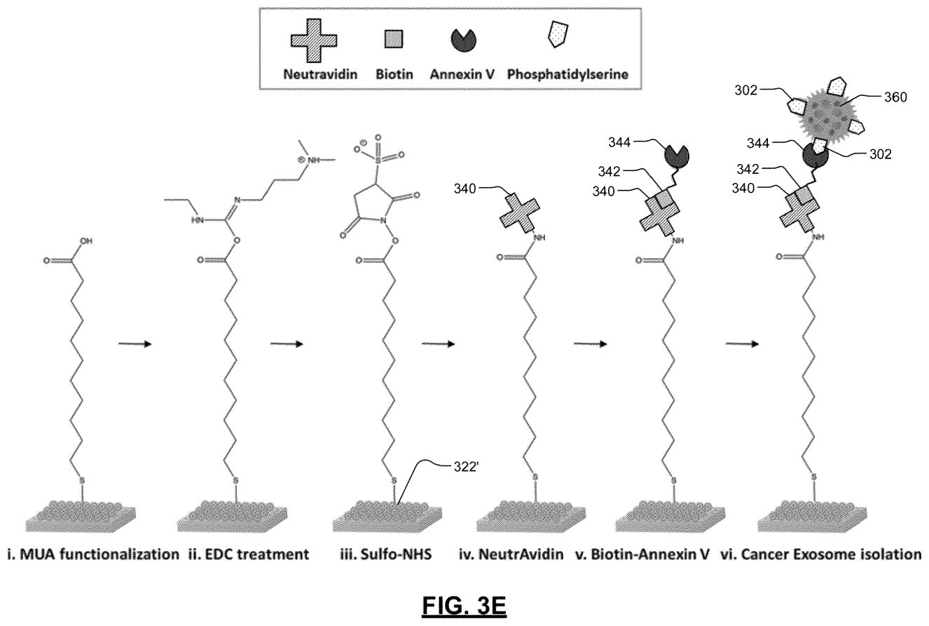

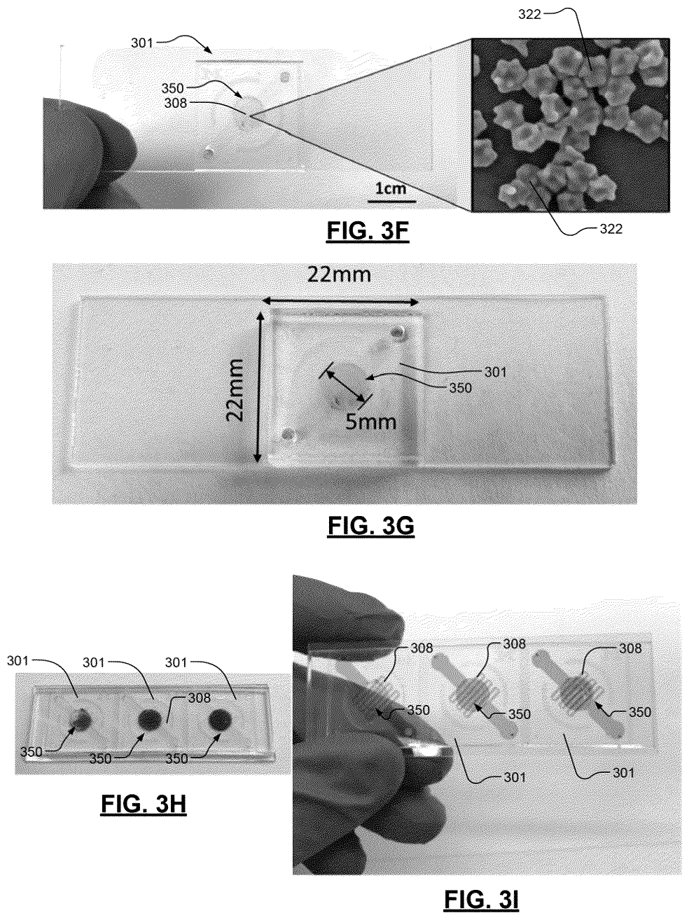

As discussed herein, chiral nanoparticles, such as gold nanoparticles (AuNPs) with an engineered chiral shape (FIGS. 2A-2L) can be integrated into a microfluidic platform that make possible rapid and robust detection of cancerous exosomes directly from plasma without prior purification steps, which is highly desirable for clinical settings (FIGS. 3A-3I). In certain variations, this can be accomplished by taking advantage of high affinity between cancerous exosomal lipid, phosphatidylserine (PS), and Annexin V associated with (e.g., anchored on) chiral AuNPs. Abundance of lipids in an exosome membrane increases efficiency of cancerous exosome capture via PS-Annexin V binding compared to previous standard isolation methods, which is needed to reduce both time and sample losses. In certain aspects, the maximum of polarization rotation in the red part of the spectrum characteristic of AuNPs affords realization of this process in microfluidic device with PDMS/glass components transparent for this part of the electromagnetic spectrum. Furthermore, the chiroptical response of the chiral assemblies in exosomes is enhanced in proximity of plasmonic nanostructures. Combined with versatility and low cost of microfluidic chips, the detection of CD signature of exosomes in microfluidics, referred to herein as Circular Dichroism-based Exosome (CDEXO) chips by use of chiral AuNPs is a convenient platform for clinical applications, such as liquid biopsy.

In various aspects, the present disclosure further contemplates methods of forming a microfluidic device (See, e.g., the microfluidic device 301 of FIGS. 3A-3B and 3F-3I). In certain variations, chiral gold nanoparticles can be incorporated into a layer-by-layer assembled coating on a channel found in the microfluidic device. In certain aspects, a microfluidic channel may be a groove or enclosed capillary optionally having a volume of less than or equal to about 30 microliters (μL). In certain variations, each microfluidic channel may have a volume of greater than or equal to about 4 μL to less than or equal to about 30 μL. The microfluidic channel may have an inner diameter of less than or equal to about 1 mm, for example, having an inner diameter of greater than or equal to about 5 μm to less than or equal to about 1,000 μm (1 mm).

The substrate may be formed of an inorganic material or a polymeric material and is desirably transmissive to electromagnetic radiation, such as circularly polarized light, in a target range of wavelengths (e.g., red light or NIR). Thus, the substrate on which the microfluidic channel is formed may comprise a material that is transparent to certain predetermined wavelengths of light, such as a silicon dioxide material (e.g., fused silica or glass or borosilicate), quartz and polymers (e.g., polycarbonate, or acrylates). The substrate may be coated, for example, with polydimethylsiloxane (PDMS). Further, the substrate may be treated, for example cleaned and/or etched (with chemicals, plasma, electron beam, or high intensity lasers, for example). In certain aspects, the substrate may be a microchip.

The chiral nanoparticles are capable of rapidly isolating and profiling cancer-associated exosomes directly from blood plasma using their own unique chiral signal. Exosomes from lung cancer patients can be distinguished from those from healthy donors by chiroptical spectroscopic signatures of biomolecular components of exosomes enhanced by chiral plasmonic nanoparticles. Furthermore, mutation/deletion of epidermal growth factor receptor also demonstrates an ability for in-depth mutation profiling in addition to cancer diagnostics. Where the microfluidic device is on a chip, it can be mounted to a conventional CD spectrometer and its measurement is simple and completed rapidly. In certain non-limiting aspects, the microfluidic device may comprise a single microfluidic channel that improves the sensitivity and speed of detection by 14 times and 10 times, respectively, compared to traditional techniques. However, multiple channel complexes or arrays of microfluidic devices are also contemplated. Optically convenient near-infrared resonances of chiral nanoparticles enable in perspective the low-cost glass/plastic-based microfluidics that represents an attractive pathway for rapid and versatile profiling of various extracellular vesicle.

In various aspects, chiral nanoparticles may be formed. (See, e.g., the chiral nanoparticles of FIGS. 2A-2L). The chiral nanoparticles may be formed by the techniques described in U.S. Pat. No. 10,279,394 to Kotov et al., which describes methods to synthesize chiral enantiopure nanoparticles and nanostructures, inter alia, the relevant portions of which are incorporated herein by reference. CD signals are observed not only from a chiral ligand itself in chiral gold nanoparticles, but also from a surface plasmon resonance region, which manifests in the metal-based electronic transition in the visible region. Briefly, such chiral nanoparticles can be formed by a method of forming a chiral nanoparticle that comprises directing circular polarized light towards a nanoparticle precursor. The circular polarized light causes a photo-induced reaction of the nanoparticle precursor and further induces chirality therein, which thus forms the chiral nanoparticle. Light in the form of a plane wave in space may be linearly polarized. Light is a transverse electromagnetic wave, but natural light is generally unpolarized with all planes of propagation being equally probable. Circular polarized light (CPL) typically has two perpendicular electromagnetic waves of equal amplitude and 90° difference in phase and may have either a left-handed orientation (where the electric vector of light originating from a source appears to rotate clockwise) or a right-handed orientation (where the electric vector of light originating from a source appears to rotate counter-clockwise). Circular polarized light may be produced by passing linearly polarized incident light through a quarter-wave plate at an angle of 45° to the optic axis of the plate, for example. Elliptical polarized light is light that has two perpendicular waves of unequal amplitude that differ in phase by 90°. In certain alternative aspects, elliptical polarized light may be used to induce chirality similar to circular polarized light.

Chirality of a nanoparticle means that a nanoparticle or nanostructure exhibits asymmetrical optical activity with different handedness (clockwise to form left handed chirality (S or L orientation) and counter-clockwise to form right handed chirality (R or D orientation). By directing circular polarized light at the precursor(s) material capable of absorbing and retaining polarization information of incident photons, it is believed that a templating process occurs as described in U.S. Pat. No. 10,279,394 to Kotov et al. Such templating appears to convert the spin angular momenta of photons into structural changes in matter, thus causing inducement of chirality in nanoparticles by enantioselective photo activation, followed by a reaction (e.g., photo oxidation) and self-assembly to form structures exhibiting chirality. If the circular polarized light directed towards the nanoparticle precursor is a left-handed circularized polarized light, the chiral nanoparticle is templated to display a left-handed chirality. If the circular polarized light is a right-handed circularized polarized light, the chiral nanoparticle displays a right-handed chirality.

Generally, a “nanoparticle” is a solid or semi-solid material that can have a variety of shapes or morphologies and may include nanostructures or assemblies of nanoparticles. However, a nanoparticle is generally understood by those of skill in the art to mean that the particle/structure has at least one spatial dimension that is less than or equal to about 10 micrometers (μm) (10,000 nm). In certain variations, a nanoparticle's longest dimension is less than or equal to about 5 μm. In certain aspects, a nanoparticle has at least one spatial dimension, such as length, that is greater than or equal to about 2 nm and less than or equal to about 5 μm, optionally greater than or equal to about 2 nm and less than or equal to about 3 μm, optionally greater than or equal to about 2 nm and less than or equal to about 1 μm, optionally greater than or equal to about 2 nm and less than or equal to about 500 nm, optionally greater than or equal to about 2 nm and less than or equal to about 100 nm. It should be noted that other dimensions might be greater than these ranges. In certain embodiments, the chiral nanoparticles may have a triangular nanoplate shape, a nanocube shape, a nanorod, nanoribbon, nanopyramid, nanoprism, nanohelix/nanohelices, twisted meshes, distorted lattices, nanobowties, nanopropellers, or a nanoassembly of shapes. In certain aspects, the chiral nanoparticles may be formed from chiral nanoparticles with near-IR activity, including active helix/helices, twisted ribbons, bowties, nanoparticles, and the like. The chiral nanoparticles may have a nanoplate, nanocube, bowtie or propeller-like shape in certain variations. In certain variations, the nanoparticles may have a cubic shape with twisted sides. In yet other variations, the complex shape of the nanoparticles may include nanorods, nanoribbons, nanopyramids, nanoprisms, nanohelices twisted meshes, cages, distorted lattices, and others.

The nanoparticle precursor used to form the chiral nanoparticle may comprise an element selected from the group consisting of: gold, cadmium, silver, copper, nickel, iron, carbon, platinum, silicon, mercury, lead, molybdenum, iron, and combinations thereof. It is desired that such precursor materials are capable of absorbing light so that they may undergo a photo activation reaction, as well as light induced self-assembly. The chiral nanoparticle may thus comprise or be formed from a light-absorbing material. In certain aspects, the chiral nanoparticle formed in accordance with certain aspects of the present disclosure is selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof. In certain other aspects, the chiral nanoparticle formed in accordance with certain aspects of the present disclosure is selected from the group consisting of: Au, CdTe, CdSe, CdS, and combinations thereof. In other alternative variations, the nanoparticles can be selected from the group consisting of: gold, silver, copper, nickel, carbon, as well as a variety of semiconductors, including direct and indirect band gap semiconductors including those listed above, and any combinations thereof.

The methods described in U.S. Pat. No. 10,279,394 to Kotov et al. form chiral nanoparticles/nanostructures without the need for any ligands as a means to induce chirality in the nanoparticle. However, chiral NPs leading to nanostructures with different chirality can be obtained by using a stabilizing or shape-directing ligand comprising D-cysteine or L-cysteine, by way of example. L-cysteine imparts clockwise, or left-handed mirror-asymmetry (FIGS. 2A and 2G-2H), while D-cysteine yields NPs with counterclockwise, or right-handed sense of rotation (FIGS. 2B and 2I-2J). Other stabilizing or shape-directing ligands may include L- or D-penicillamine, dopamine, L-carnosine, amyloid peptide monomers, amino-acid derivatives, and any combinations thereof.

The nanoparticle precursor may be a racemic mixture prior to exposure to the circular polarized light, whereas after the treatment, an enantiomeric mixture having greater than or equal to about 25% enantiomers and optionally greater than or equal to about 30% enantiomers is produced. In certain variations, the nanoparticle precursor comprises a first component for forming the chiral nanoparticle and a second component that serves as a capping agent on the chiral nanoparticle. A suitable capping agent may be an achiral capping agent, such as thioglycolic acid (TGA). The nanoparticle precursor is desirably a dispersion of a first component, and optionally of the second component, for forming the chiral nanoparticle in an aqueous medium. Such nanoparticle dispersions are desirably stable at ambient conditions.

CPL “templating” of NP assemblies is based on the enantioselective photo activation of chiral NPs and clusters, followed by their photooxidation and self-assembly into nanoparticles with specific helicity because of chirality-sensitive interactions between the NPs. Thus, these chiral NPs have the ability to retain the polarization information of incident photons. The chiral nanoparticle formed in accordance with various aspects of the present disclosure is stable and maintains its chiral properties for greater than or equal to about 1 year.

In certain variations, the chiral NPs are capable of having a strong adhesion to both the substrate and sEVs. A large contact area with the surface of a macroscale fluidic channel and nanoscale spherical sEVs imposes contradictory geometrical requirements on the NP assemblies which can be resolved using complex chiral geometry. In certain variations, a chiral AuNP may be formed from triangular nanoplate precursors that have strong attractive interaction with flat surfaces, such as a strong attractive interaction with the substrate surface. Chiral features on these initial NPs are grown by adding gold precursor, L-ascorbic acid as a reductant, and L- or D-cysteine (Cys) as a shape-directing agent.

In certain aspects, the chiral NPs include a plurality of vertices. In certain aspects, the chiral NPs include at least one concave region, or pocket, adjacent to the vertices. For example, with reference to FIGS. 2G-2J, AuNPs 202, 204 include a plurality of vertices 206 and a plurality of concave regions 208. In certain aspects, each of the concave regions 208 has at least one spatial dimension, such as a diameter, that is greater than or equal to about 5 nm to less than or equal to about 100 nm, optionally greater than or equal to about 10 nm to less than or equal to about 90 nm, optionally greater than or equal to about 20 nm to less than or equal to about 80 nm, optionally greater than or equal to about 30 nm to less than or equal to about 70, or optionally greater than or equal to about 40 nm to less than or equal to about 60 nm. It should be noted that other dimensions might be greater than these ranges. The plurality of concave regions 208 may appear at an intermediate stage of chiral AuNP formation, as chiral AuNPs including the concave regions 208 may exhibit a relatively slower growth rate in a first, or vertical direction, as compared to a growth rate of chiral AuNPs free of concave regions. In certain aspects, AuNPs including concave regions 208 exhibit improved surface modification and/or functionalization abilities as compared to AuNPs that are free of concave regions. In certain aspects, the AuNPs including concave regions 208 may have a higher affinity of thiol functional groups, such as a mercaptoundecanoic acid (MUA), as compared to AuNPs free of concave regions. In certain aspects, AuNPs exhibiting increased specialized surface modification, such as surface modification with MUA, improve adhesion between the AuNPs and the sEVs. Furthermore, AuNPs including the plurality of concave regions 208 may provide relatively higher absorbance and scattering of incident electromagnetic waves as compared to AuNPs that are free of concave regions.

In certain aspects, the nanoparticles are encapsulated and stabilized by positive charged bilayer micelles, for example, cetrimonium bromide (CTAB)—a quaternary ammonium surfactant, thus avoiding aggregation during the metal reduction.

In certain aspects of the present disclosure, the methods may include forming a microfluidic device. In certain aspects, a detailed schematic view of a CEDXO microfluidic device 301 (FIGS. 3A-3B and 3F-3I) for detecting a bioactive target analyte 302 in a biological fluid sample (such as blood or plasma) obtained from a subject is shown. In certain aspects, the bioactive target analyte 302 is associated with an exosome or sEV (FIGS. 3A and 3E). With reference to FIG. 3C, as shown in block 303, a method 300 may be a layer-by-layer (LBL) process that optionally comprises treating a substrate 304 to have a first polarity. As shown in block 305, the method optionally comprises applying a first charged material 306 having a second polarity to at least one surface 308 of a microfluidic channel 310 on the substrate 304 having the first polarity opposite to the second polarity. As shown in block 312, the method also includes applying a second charged material 314 having the first polarity over the first charged material 306 in the layer-by-layer (LBL) process on the at least one surface 308. The first charged material 306 and the second charged material 314 are distinct from one another and define at least one layer of a mutilayered coating 316 (having alternating layers of the first charged material and the second charged material) (FIG. 3D). Although only one layer of the multilayer coating 316 is shown in FIGS. 3C-3D, the microfluidic device may comprise more than one multilayered coating 316.

The LBL technique is well known and relies on alternating adsorption of charged species or polyelectrolytes onto a substrate. Layers may be built up by sequential dipping of a substrate into oppositely charged solutions having oppositely charged moieties that are attracted to the surface. Additional steps may occur between application steps, such as washing of the surface before application of the next material. Monolayers of individual components attracted to each other by electrostatic and van-der-Waals interactions are thus sequentially adsorbed on the substrate. Multiple deposition cycles of first and second charged materials can be repeated sequentially to build alternating layers in a multilayered structure. A layered material formed by LBL is often referred to as: (polyanion/polycation)ₙ, where n represents the number of deposition cycles or layers present. LBL films or coatings can be constructed on a variety of solid substrates, thus imparting much flexibility for size, geometry and shape and further patterned or etched (with chemicals, plasma, electron beam, or high intensity lasers, for example).

In certain aspects, as shown in block 303 (FIG. 3C), the substrate 304 (FIGS. 3C-3D) may have a negative charge or may be treated to impart a negative charge. In one example, the substrate 304 is a glass substrate. In certain aspects, the substrate 304 may include a charged material or moiety and may be cleaned, for example, with a Piranha solution followed by a plasma treatment 318 (e.g., oxygen plasma treatment). In certain aspects, after the plasma treatment 318, the glass substrate 304 has a negative charge 319 (FIG. 3D). In other aspects, the substrate 304 may be treated with other chemicals, electron beam, or high intensity lasers, for example.

As shown in block 305 (FIG. 3C), the first charged material 306 may be applied over the negatively charged substrate 304 (FIGS. 3C-3D). In certain aspects, the first charged material 306 may be a polycation. In certain aspects, the first charged material 306 may be a poly(dimethyldiallylammonium chloride) (PDDA), a polyurethane (PU), or combinations thereof.

Then, as shown in block 312 (FIG. 3C), a second charged material 314 may be formed over the first charged material 306 (FIGS. 3C-3D). In certain aspects, the second charged material 314 may be a polyanion. In certain aspects, the second charged material 314 may be an anionic polystyrene sulfonate (PSS).

Next, as shown in block 320 (FIG. 3C), the method 300 comprises applying a plurality of chiral nanoparticles 322 over the layered coating 316 (FIG. 3D), so that the plurality of chiral nanoparticles 322 are exposed to the microfluidic channel 310 (FIG. 3C). Each of the chiral nanoparticles 322 comprises a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof. In certain aspects, the chiral nanoparticles 322 comprise gold and are positively charged. Thus, the cationic chiral gold nanoparticles 322 are deposited over the second charged anionic material 314.

According to various aspects of the present disclosure, the methods may also include functionalizing the plurality of chiral nanoparticles and associating each nanoparticle of the plurality with a targeting ligand that is capable of binding to the bioactive target analyte in the biological fluid sample. In certain aspects, the bioactive target analyte indicates a presence of cancerous cells or mutated proteins in the biological fluid sample.

As shown in block 330 (FIG. 3C), in certain aspects, the method 300 optionally includes functionalizing the plurality of chiral nanoparticles 322. In certain aspects, the plurality of chiral nanoparticles 322 comprise chiral gold nanoparticles and the first aspect of the functionalizing comprises reacting the chiral gold nanoparticles with MUA. Next, the MUA is reacted with 1-ethyl-3-(3-dimethylaminopropyl)carbodiimide (EDC). Finally, the functionalizing may include reacting with N-hydroxysuccinimide (NHS) with the EDS to form a plurality of functionalized chiral gold nanoparticles 322′.

As shown in box 332, the functionalized nanoparticles 322′ may then be reacted with one or more moieties or targeting ligands 334 for reacting with the bioactive target analyte 302. For example, the targeting ligand 334 may be an antibody that binds with the bioactive target analyte 302. In one variation, the targeting ligand 334 may comprise Annexin V cellular protein with the capability of binding to PS. Other targeting ligands may be anti-CD63, anti-CD81, anti-CD9, anti-CD56, anti-CD-133 (tetraspanin proteins) targeting extracellular vesicle (EV) surface proteins, anti-EpCAM, anti-EGFR, anti-vimentin, and the like. In one variation, where the targeting ligand 334 is Annexin V, it may be further complexed or associated with other ligands. For example, as shown in FIG. 3E, the functionalized gold nanoparticle 322′ is complexed with NeutrAvidin protein 340, which is deglycosylated native avidin from egg whites. Next, the NeutrAvidin 340 is associated with biotin 342. The biotin 342 then is associated or complexes with Annexin V 344, which is capable of binding to the bioactive target analyte 302 (in this case PS).

As such, with renewed reference to FIGS. 3A-3B and FIGS. 3F-3I, the present disclosure provides the microfluidic device 301 that includes the microfluidic channel 310 defining an inlet, an outlet, and at least one sensing region 350. In certain aspects, each of the microfluidic devices 301 comprises the at least one surface 308 having the plurality of chiral nanoparticles 322 disposed thereon. The plurality of chiral nanoparticles 322 each comprises a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof. In certain variations, gold chiral nanoparticles. The chiral nanoparticles 322 also include or are functionalized by a targeting ligand 334 associated with the plurality of chiral nanoparticles 322 that is capable of binding to a bioactive target analyte 302 in abiological fluid sample. As discussed above, the bioactive target analyte 302 indicates apresence of cancerous cells or mutated proteins in the biological fluid sample. In certain aspects, AuNPs are engineered to have both strong adhesion to the substrate 304 of the microfluidic channel 310 and concurrently to exosomes 360. This may be done by optimizing particle size and surface coating in the stages of the particle growth and subsequent functionalization (see by way of example, the techniques described in Wei Ma, et al., “Chiral Inorganic Nanostructures,” Chem. Rev., 117 (12), pp. 8041-8093 (2017), the relevant portions of which are incorporated by reference. In some implementations, it can also be accomplished by using illumination with circularly polarized light, such as described in U.S. Pat. No. 10,279,394 to Kotov et al.

The microfluidic channel 310 optionally comprises the multilayered coating 316 formed by a LBL deposition process that comprises a plurality of positive layers and a plurality of interspersed negative layers. An exposed surface of the multilayered coating 316 comprises the plurality of chiral nanoparticles 322 having a positive charge.

In certain aspects, the mutlilayered coating 316 comprises at least one layer of the plurality of positive layers comprises a cationic PDDA and at least one layer of the plurality of negative layers comprises an anionic PSS.

In certain other aspects, the at least one surface 308 of the microfluidic channel 310 is plasma etched. The microfluidic channel 310 may be formed on a microchip. In certain variations, the bioactive target analyte 302 comprises PS and the the targeting ligand 334 comprises Annexin V. In certain variations, the plurality of chiral nanoparticles 322 comprise chiral gold nanoparticles functionalized with MUA reacted with EDC and NHS. The targeting ligand 334 may further comprise deglycosylated avidin associated with biotin that is associated with Annexin V.

The microfluidic device 301 may include other conventional components not shown, including seals, gaskets, flow regulators, pumps, valves, ports, manifolds, sensors, monitors, and the like. The microfluidic device 301 may be associated with a circular spectroscopy device and one or more detectors. The microfluidic device 301 may further include a control system for automated operation, which may be a microprocessor or a computer processing unit (CPU).

As discussed above, the present disclosure also contemplates methods of detecting a target bioactive analyte in a biological fluid sample obtained from a subject, for example as discussed above in the context of FIG. 1. Such a method may be conducted on a microfluidic device like that shown in FIGS. 3A and 3B, as discussed herein. The method comprises passing a biological fluid sample through a microfluidic channel) comprising the at least one surface 308 having the plurality of chiral nanoparticles 322 disposed thereon. The plurality of chiral nanoparticles 320 each comprises a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof and a targeting ligand that is capable of binding to the bioactive target analyte 302 optionally present in the biological fluid sample. The biological fluid potentially containing the bioactive target analyte 302 contacts the plurality of chiral nanoparticles 322 and thus any bioactive targeting analyte 302 can bind with the targeting ligand 334 on the chiral nanoparticles 322. The method includes directing circularly polarized light 362 at the microfluidic channel 310 while the biological fluid sample is disposed in the microfluidic channel 310 to measure a first level of at least one of circular dichroism or wavelength. The measuring may occur within 10 minutes to one day within introducing the biologican fluid sample into the microfluidic channel 310. As shown in FIG. 3B, a first level peak 370 is shown as a first line 372 at about 580 nm.

The method further includes comparing the first level of at least one of circular dichroism or wavelength to a baseline level. In certain aspects, a second, or baseline, level peak 373 is shown as a second line 374 in FIG. 3B of at least one of circular dichroism or wavelength in the microfluidic channel 310 in the absence of the biological fluid sample. A measured difference 376, depecited as Δλ, between the first level peak 370 and the baseline level peak 373 indicates a presence of the bioactive target analyte 302, which in certain variations may indicate a presence of cancerous cells or mutated proteins in the biological fluid sample. As shown in FIG. 3B, the baseline level peak 373 has a lower a.u. intensity for CD and a peak wavelength of about 555 nm, while the first level peak 370 is at about 580 nm or a spectral or wavelength shift in peaks of approximately 25 nm. Further the peak CD intensity is greater for the first level peak 370 than the baseline level peak 373. A measured difference 378, depecticted as ACDfurther indicates the presence of the exosomes 360 binding with the targeting ligands 334 on the chiral nanoparticles 322 in the microfluidic channel 310. In this manner, the present disclosure contemplates methods for detecting one or more bioactive target analytes 302 that may indicate the presence of cancer or mutated proteins from a subject by processing a biological sample obtained from the subject.

In certain aspects, the method may also provide an ability to profile cancer exosomes from the biological fluid sample. In certain aspects, the profiling provides an ability to quantify how much target analyte 302 is present in the sample because spectral shift and CD magnitude change appear to be proportional to a quantity of spiked extracellular vesicles (e.g., a quantity of the exosomes 360). Thus, it appears that proportional changes occur in spectral shift and/or CD peak magnitude relate to concentration of the bioactive target analyte 302 present in the biological fluid sample. As such, the method also optionally comprises comparing the first level/value with a baseline level value (or a table of various premeasured levels/values quantifying amounts of the target analyte present) to determine an amount of the bioactive target analyte 302 present in the biological fluid sample.

Spectra shift and CD magnitude change can be monitored using a CD-spectrometer in certain variations. However, it is also contemplated that the chiral nanoparticles 322 may be designed to have spectral shifts in the visible spectrum of light, so that detection may be made by an observer (e.g., detection can be evaluated using the naked eye by looking into color change on a microfluidic device with samples).

In certain aspects, the method may further comprise measuring the baseline level of at least one of circular dichroism or wavelength by directing the circularly polarized light 362 at the microfluidic channel 310 in the absence of any biological fluid sample. In certain variations, the first level has a peak wavelength measured in a range of greater than or equal to about 520 nm to less than or equal to about 1.4 μm, for example, in certain aspects, it may be a red light or NIR having a peak wavelength of greater than or equal to about 625 nm to less than or equal to about 1.4 μm.

The method may further comprise washing the microfluidic channel 310, for example, eluting any bound bioactive target analytes 302 from the chiral nanoparticles 322 in the microfluidic channel 310 so that the microfluidic device 301 can be reused. For example, a chelating ethylenediaminetetraacetic acid (EDTA) can be used to release and elute the bound target analytes, like exosomes, from the Annexin V in the sensing regions of the microfluidic channel.

Various embodiments of the inventive technology can be further understood by the specific examples contained herein. Specific examples are provided for illustrative purposes of how to make and use the compositions, devices, and methods according to the present teachings.

### Example

### Synthesis of Chiral Gold Nanoparticles

All glassware is pre-treated with aqua regia solution for removal of metal residues and rinsed with DI water thoroughly. Gold triangular nanoplates are optically normalized for consistent batch-to-batch concentration of AuNPs. For the preparation of the growth solution, 32 ml of 12.5 mM cetrimonium bromide (CTAB), 0.4 ml of 20 mM HAuCl₄, 4.0 ml of 0.1 M AA and 0.08 ml 0.1 mM L-cysteine (or D-cysteine) are mixed with a vortex mixer. CTAB, HAuCl₄, AA and L-cysteine act as a stabilizer, a meter precursor, a reductant and a chirality-controller, respectively. Then, 2 ml of Au nanoplate solution is added in the prepared growth solution to initiate the chiral growth on the surface of targeted AuNPs. All procedures proceed at 30° C. After 4 hours, the growth rate is saturated, and the resultant is separated by using a centrifuge and re-dispersing in DI water.

### Zeta-Potential (ξ-Potential) and Size Distribution of Chiral AuNP Probes

The ξ-potential and size distribution are measured with a Nano ZS Zetasizer instrument (Malvern Instruments, Malvern, Worcestershire, UK). For ξ-potential, samples are equilibrated for 120 seconds before each measurement; all measurements are conducted in triplicate, each measurement included 50 cycles, and a 15-s pause was included between runs.

Numerical Simulation of Optical Activities from Chiral AuNPs

To compute the optical activities of the chiral AuNPs, the frequency domain from of Maxwell's equations is solved with the finite element method (FEA) using COMSOL Multiphysics 5.5 software package (the radio frequency module). The 3D propeller shaped disk particles (FIG. 2G) are modelled based on the shape and dimensions found from electron microscopic images. The model structures are placed in a homogeneous surrounding medium with an effective refractive index of 1.33 (water) and 1.0 (air) for colloidal and chip samples, respectively. The dielectric functions of AuNP are taken from literature. The maximum mesh size of finite element analysis is set to λ/4n, where λ is the wavelength of incident light and n is the real part of the material refractive index is used to minimize the scattering from the outer boundary. The particles are placed in the x-y plane parallel to their longitudinal axis, while the circularly incident light is propagated along the z-axis with port power of 8.1×10⁻⁵ W. For colloidal particle analysis, the particle structure is rotated with step of π/6 along with x and y axis and the averaged extinction cross sections are obtained to analyze chiroptical response of the model. For analysis of solid chip samples, spectra are calculated at one angle, where the longitudinal axis of particles is normal to incident direction (z), as the model is initially placed.

The model computes the scattering, absorption, and thus extinction cross-sections of the AuNP enantiomers under LCP and RCP light.

The total scattering cross-section (σ_{sc}) is defined as

$${σ}_{s c} = \frac{I}{{I}_{0}} \int \int ( n \cdot {S}_{s c} ) dS .$$

Here, n is the normal vector pointing outwards from the local surface, S_{sc} is the scattered intensity (Poynting) vector, and I₀ is the incident intensity. The integral is taken over the closed surface of the meta-atom. The absorption cross section (σ_{abs}) is expressed as

$${σ}_{a b s} = \frac{I}{{I}_{0}} \int \int \int Q d V ,$$

where Q is the power loss density in the structure and the integral is taken over its volume.

The total extinction cross section (σₑₓₜ) is simply the sum of the scattering and absorption:

$${σ}_{ext} = {σ}_{s c} + {σ}_{a b s}$$

The chiroptical response of the particles can be characterized as σ_{ext,RCP}−σ_{ext,LCP}.

### Fabrication of Microfluidic Channels for CDEXO Chip

The top layer and bottom masking layer of a CDEXO chip is fabricated by standard soft lithography including mold fabrication and PDMS molding. By patterning SU8-2050 photoresist on a silicon wafer, the top and bottom masking layer molds are prepared. The top chamber layer is fabricated by pouring PDMS and PDMS curing agent mix (1:10) (Dow Corning, US) onto the silicon mold after degassing of PDMS mixture in vacuum for 10 minutes. The thin masking layer is prepared using a PDMS mixture spun on the silicon mold at 1,000 rpm for 30 seconds and followed by an incubation at 70° C. for 2 h. The top and bottom layers are cut, punched and placed for processing of samples.

### Chiral AuNP Deposition on Glass Substrate

A standard glass slide is treated first by piranha solution and incubated overnight to activate negatively charged functional groups on the glass surface. After gentle washing with water and undergoing a drying procedure, a thin layer of PDMS with openings is attached on the slide glass assisted by an electrostatic binding. 100 μL droplets of 5% PDDA solution are applied to each opening and allowed to incubate for 1 hour at room temperature. The devices are then washed by dipping into 4 separate tubes of DI water consecutively followed by a thorough and careful air-drying. Next, 100 μL droplets of 0.5% by weight PSS solution are applied to each opening on the glass slides and allowed to incubate for 1 hour at room temperature. After another washing step, 30 μL droplets of the prepared 10× chiral AuNP solution are applied to each opening region and allowed to incubate for 1 hour at room temperature.

### Surface Modification of Chiral AuNP for Exosome Isolation and Sensing

After incubation, the excess unbound AuNP solution is rinsed off with DI water, and the devices are placed in 0.25 mM MUA solution prepared in ultrapure ethanol for overnight incubation. In order to functionalize NeutrAvidin onto the glass surface, carbodiimide crosslinker chemistry is utilized. The devices are taken out of the MUA solution, washed with ethanol, and dried by carefully blowing air onto the surfaces. 100 μL droplets of 4 mM EDC solution prepared in DI water are applied to each sensing region and allowed to incubate for 30 minutes at room temperature. The excess EDC solution is then rinsed off with DI water, and 100 μL droplets of 8 mM Sulfo-NHS solution (diluted in DI water from 50 mM stock) are applied and allowed to incubate for 30 minutes at room temperature. Each device is rinsed in DI water, and 100 μL droplets of 0.03 μM NeutrAvidin solution (diluted in PBS from 3 μM stock) are applied and allowed to incubate overnight at 4° C. When devices are needed for sample processing, they are taken out of the 4° C. refrigerator, washed with PBS, and dried by carefully blowing air over the surfaces. 30 μL droplets of 10× diluted biotinylated-Annexin V (diluted in calcium rich 1× binding buffer) are applied and allowed to incubate for 40 minutes. This is followed by a wash with the 1× binding buffer, air dry, and CD signal reading as a baseline before the top PDMS layers are applied and samples processed.

### Cell Culture and Cell-Derived Exosome Preparation

As model samples, exosomes from three different lung cancer cell lines (A549, H1650 and H3255) and one lung fibroblast cell line (MRC5) are prepared. Lung cancer cell lines are cultured in serum free media for 3 days, and the cell culture supernatant is centrifuged at 2,000×g for 15 minutes, the resultant supernatant is followed by a second centrifugation at 12,000×g to remove all residual cellular debris. The supernatant is then ultracentrifuged at 100,000×g to isolate exosomes. Each cell line is cultured in conditioned media with exosome depleted fetal bovine serum (FBS) for 1-3 days and the cell culture supernatant are ultracentrifuged to isolate exosomes. After exosome separation, the concentration of samples is measured using nanoparticle tracking analysis (NTA), and a known number of exosomes was used for model sample preparation.

### Human Plasma Sample Preparation

The clinical sample collection and experiments are approved by Ethics committee (Institutional Review Board and Scientific Review Committee) of the University of Michigan. Informed consent is obtained from all participants of this clinical study and the blood samples of cancer patients are obtained after approval of the institutional review board at the University of Michigan (HUM00119934). All experiments are performed in accordance with the approved guidelines and regulations by the ethics committee at the University of Michigan. For plasma separation from whole blood, each blood sample is centrifuged using 5810R centrifuge (Eppendorf, Germany) at 2,000×g for 15 minutes to sediment all nucleated cells and followed by second centrifugation at 12,000×g to remove all residual cellular debris. The clear supernatant from the second centrifugation is gently collected, filtered through a 200 nm syringe filter and used in the study thoroughly.

### Sample Preparation and On-Chip Processing

As the Annexin-V-based exosome isolation is calcium dependent, all samples are prepared in a calcium containing buffer (see, e.g., the Ca²⁺380 of FIGS. 3A-3B). For model samples, pre-isolated cell-derived exosomes are spiked into the 30 μl of 1× binding buffer containing 2.5 mM of CaCl₂. The plasma samples are also mixed with 10× binding buffer, so that the mixture has the equivalent Ca²⁺ ions concentration to model samples. 30 μl of sample is injected to CDEXO chip through the inlet and incubated for 20 minutes. After exosome capture, 100 μl of 1× binding buffer is flowed to remove excess unbound vesicles or protein debris in plasma. For exosome release from the chip, 100 μl of 20 mM EDTA solution is flowed and followed by injection of 100 μl of PBS buffer. The collected samples from the device underwent further NTA analysis depending on the study. In every step, spectral signal of the CDEXO chip under CD spectrometry is performed and evaluated.

### Circular Dichroism Spectrometry Analysis

Circular dichroism (CD) spectra are obtained using a Jasco J-815 CD spectrometer. Jasco J-815 is also used for CD spectra measurement of model exosomes in a quartz cuvette. For CDEXO chip experiment, the CD signal is taken in between each step of the sample processing procedure, including baseline, post-capture, and post-release of exosomes. The baseline is indicative of the signal given off by the NPs, which are synthesized with L-Cys on their surfaces as well as functionalized with the NeutrAvidin and Annexin V required to capture exosomes. The bulk of this baseline signal can be attributed to the optically active L-Cys, although there are observable minimal shifts throughout the functionalization procedure that are monitored by CD. In every step, the CD spectra measurement is taken after removing the top PDMS layer of CDEXO, washing with buffer, and mild air-drying. The bottom layer of CDEXO chip is adhered to CD spectrometry insert using adhesive tapes and each measurement is conducted in triplicate.

Nanoparticle Tracking Analysis of Exosomes from Cancer Cells

For the evaluation of the concentration and the size distribution of the resultant effluent, nanoparticle tracking analysis (NTA) is performed using the NanoSight NS300 (Marven Instruments, UK). For each measurement, 30 μL of the resultant is used and a laser module is mounted inside the main instrument housing. NTA visualizes the scattered lights from the vesicles of interest based on their Brownian motion. This movement is monitored through a video sequence for 20 seconds in triplicate. All data acquisition and processing are performed using NanoSight NS300 control software (screen gain, 7; camera level, 13; detection threshold, 5) and concentration of particles in exosome sizes (30-150 nm) is used for calculating capture efficiencies of the present technology.

### Cryo-SEManalysis for Exosome Sensing

The surface of CDEXO chip with or without exosome captured is examined by Helios FIB SEMwith cryo-stage at −180° C. under beam energies (2.0-5.0 kV) at the Michigan Center for Materials Characterization at University of Michigan. The cleaned silicon wafer is used as the substrate Right after exosome capture experiments, CDEXO chip substrate are naturally dehydrated. The dehydrated specimen is then mounted on an SEM stub and imaged on the cryo-stage to reduce damage of exosomes.

### Electromagnetic Simulations for Spatial Light-Matter Interaction Evaluation

The spatial map of absorbance, scattering, and extinction are collected using similar COMSOL system as described above. The 3D structure of NP with concave regions (diameter=50 nm) (see, e.g., concave regions 308 of FIGS. 2G-2J) are placed under both CPLs with the wavelength at 550 nm. The integral projection of absorbance is evaluated by using global projection function to project 3D total energy dissipation density map on to the surface of NP model. The complex model with exosome-mimic dielectric particle were constructed with spherical particle with 24 nm radius with refractive index of 1.38, which corresponds to the refractive index of exosome in literature.

### Western Blot Analysis

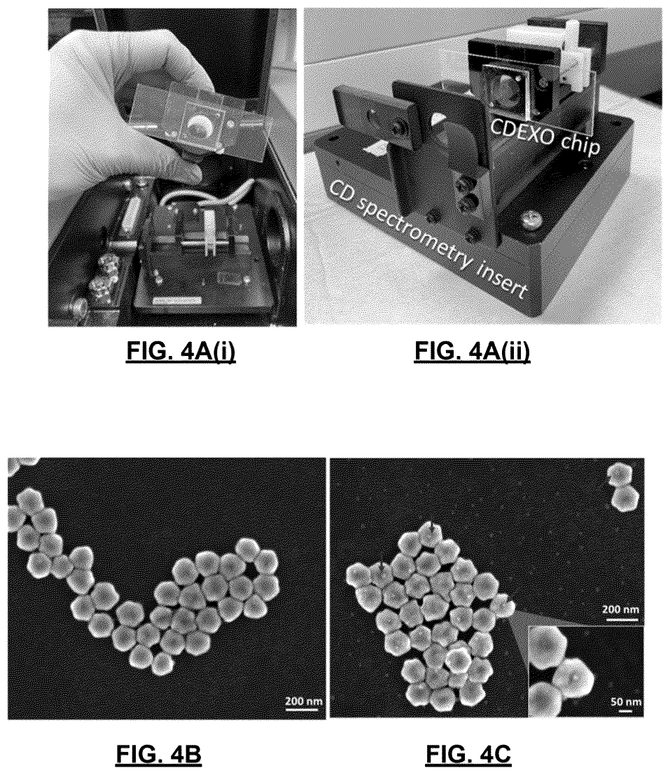

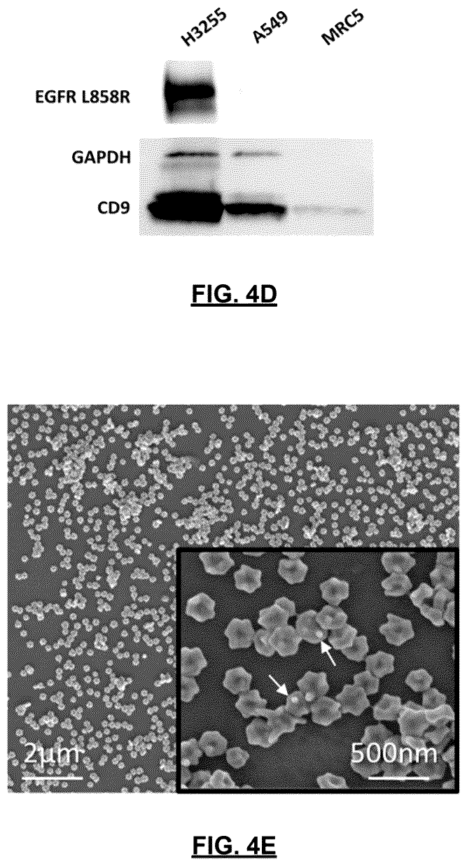

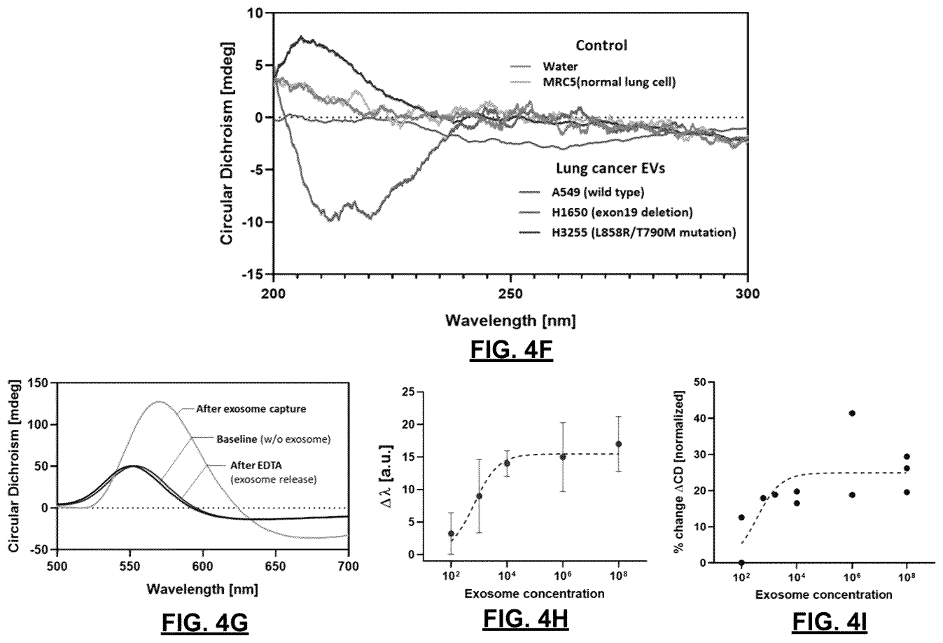

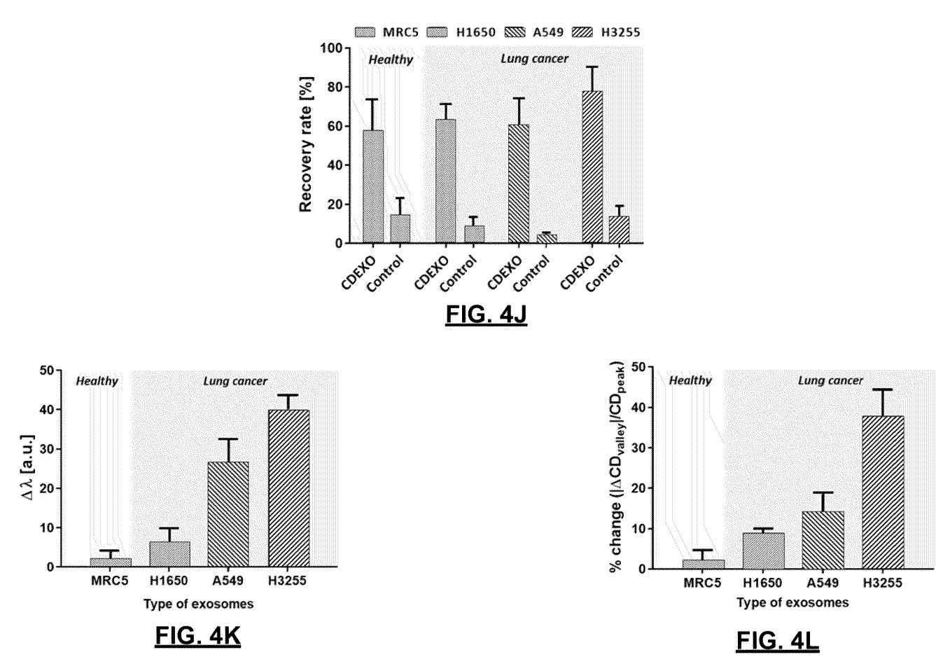

The RIPA buffer with 1% protease inhibitor is prepared for captured exosome lysis. The prepared buffer solution is applied to exosome samples. Total amount of proteins is measured by standard BCA analysis according to the manufacturer's instructions. Western Blot analysis is performed on a precast 4-20% SDS gel from BioRad (FIG. 4D). The samples are prepared in 4× Laemelli buffer with 2-mercaptoethanol and heated to 90° C. for 15 minutes before loading onto the gel. The gel is run at 120V for 1 hour before transferring at 120V for 1 hour 15 minutes on ice. Blocking is performed in 5% non-fat milk in TBST for 90 minutes. Primary antibody incubated overnight on a rocker at 4° C. at a concentration of 1:1000 in 3% non-fat milk in TBST. Thorough rinsing is performed, and then secondary antibody is incubated for 90 minutes at room temperature at 1:500 in 3% non-fat milk in TBST.

### Statistical Analysis

All results present as mean±standard deviation. Statistical analysis are demonstrated using Prism software. Unpaired t-tests (two-tailed) are used to compare the differences between peak shifts (Δλ) and % changes of magnitude change (ΔCD) in lung cancers versus healthy controls. The same statistical test is used for magnitude change and % change comparison in EGFR deletion/mutation subgroups. Statistical significance is defined as a two-tailored p<0.05.

Chiral nanoparticles for exosome sensing in the form of gold nanoparticles (AuNPs) are engineered to have the strong adhesion to the substrate and exosomes at the same time, as discussed above in the context of the nanoparticle shapes. Large contact area with the surface of a macroscale fluidic channel and nanoscale spherical exosomes imposes contradictory geometrical requirements on the biofunctionalized chiral nanoparticles, which can be resolved using complex chiral geometry at the scale comparable to exosomes to increase their overall binding affinity. Synthesis begins from gold triangular nanoplates that have strong attractive interaction with the flat surfaces. Chiral features on these initial NPs are grown by adding gold precursor, L-ascorbic acid as a reductant, and L- or D-cysteine (Cys) as a shape-directing agent. NPs are encapsulated and stabilized by positive charged bilayer micelles, CTAB, avoiding aggregation during the metal reduction.

Handedness of Cys determines nanoscale chirality of the resulting propeller-like NPs (FIGS. 2A and 2B). L-Cys imparts clockwise, or left-handed mirror-asymmetry, while D-Cys yields NPs with counterclockwise, or right-handed sense of rotation. Microscopy data about intermediate stage of AuNP growth (upper inset) indicate that chiral shape of the NPs can be controlled to optimize binding of nanoscale biomolecules, their assemblies, and extracellular vesicles. AuNPs synthesized with both L- and D-Cys have the same UV-vis absorbance spectra, while their CD spectra are nearly perfect mirror images of each other (FIGS. 1C-1E), that can also be used for affinity optimization.

The chiroptical properties of AuNPs are modeled to detail the origin of peaks in the CD spectra. Using the methodology developed previously, the three-dimensional shapes of AuNPs observed by electron microscopy (FIGS. 2G-2J) are imported into COMSOL computational environment for calculation of their optical properties (FIG. 2K). The calculated CD spectra, i.e., the difference between extinction cross sections for left-handed and right-handed circularly polarized light (σLCP—σRCP), match well with the experimentally observed chiroptical bands for both its spectral range and signs. The chiroptical activity of AuNP films assembled on the surfaces of the microfluidic channel of the CDEXO chips are calculated using the same technique also matched the experiments (FIG. 2L).

### CDEXO Chip Design for Exosome Sensing

The CDEXO chip has one sample inlet and outlet and a sensing region where chiral AuNPs are deposited (FIGS. 3A-3B). A layer-by-layer (LBL) assembly method is used to deposit chiral AuNPs on the bottom glass layer of the device, because this method affords uniform coating regardless of complexity of the geometric shapes of the substrate. The glass slide with a masking layer of PDMS is originally negatively charged by piranha and plasma treatment, followed by deposition of alternative multilayer films of cationic poly(dimethyldiallylammonium chloride) (PDDA) and anionic polystyrene sulfonate (PSS). The chiral AuNPs with positive surface charges enable AuNPs affixed to the final layer of anionic PSS on the pretreated glass surface (FIGS. 3C-3D). The AuNPs are exposed to the open volume of the microfluidic channel so that they may interact with passing fluids.

Successful AuNP deposition is further evaluated using Scanning Electron Microscopy (SEM). AuNPs are spread across the device's surface with minimal aggregation, leaving empty spaces of varying sizes between some aggregates. The surface of chiral AuNP deposited onto slide glass is modified or functionalized by treating with 1-ethyl-3-(3-dimethylaminopropyl)carbodiimide (EDC) and N-hydroxysuccinimide (NHS) chemistry, and Annexin V molecules are further immobilized on the AuNPs (FIGS. 3C and 3E).

The design of CDEXO chip allows for a direct use of conventional CD spectrometry (FIGS. 4A(i) and 4A(ii)). Once the exosomes are injected into the CDEXO chip (FIG. 3B), the characteristic CD peak of chiral NPs undergoes spectral shift (Δλ) and a magnitude change (ΔCD). Using these two parameters results from exosome binding, the microfluidic device both detects and profiles specific exosomes bound to the device surface. Due to biocompatibility and intracellular stability of AuNPs, chiral AuNPs have been used for intracellular molecule detection, such as telomerase or adenosine-5′-triphosphate (ATP). However, exosomes are significantly smaller than cells, so it is hypothesized that the chiral AuNP probe will not penetrate into exosomes, but rather bind together and offer characteristic bands under circularly polarized light.

### Characterization of Model Exosomes

For an initial evaluation of the device in exosome isolation and profiling, three different sources of exosomes are used; those secreted from normal lung fibroblasts (MRC5), lung cancer cells with wild type epidermal growth factor receptor (EGFR) (A549), lung cancer cells with exon 19 deletion (H1650) and lung cancer cells with EGFR L858R point mutation (H3255). These four different exosomes are first analyzed in western blot analysis, which quantifies protein expression in exosomes. All exosome samples from the different cell lines samples displayed CD9, a widely used exosome marker. Using two variants of a lung cancer-associated protein, total EGFR and L858R mutant EGFR, it is found that only two of the cancer cell-derived exosome samples express EGFR and not MRC5, and the L858R mutation is specifically associated with H3255 cell derived exosomes, which the cells are known to carry (FIG. 4B).

Prior to the CD analysis of these exosomes using CDEXO chip, the same amount of exosomes in water are prepared and their CD spectra is obtained (FIG. 3C). First, the region between 200 nm to 300 nm are analyzed, in which the fluctuations of the curves from the baseline seem to align with those characteristics of different types of secondary protein structures. It is apparent that exosomes derived from A549, wild type EGFR, contain large density of alpha helices, whereas exosomes derived from H3255, which shows L858R EGFR mutation, contain proteins with random coil structures.

Conversely, the normal lung fibroblast sample, MRC5, and the exon 19 deletion lung cancer exosomes, H1650, match almost perfectly with the CD spectra from the baseline used to compare these samples, showing no alignment with any fluctuations shown by secondary structures. It is unclear whether these results are indicative of the major secondary structures present on the surface of these exosome samples, but they are nonetheless useful for the purpose of characterizing different exosome samples using the present technology. The results demonstrate that the cancer cell-derived exosomes have specific chiroplasmonic signature measurable by CD spectroscopy. Additionally, the results indicate that each exosome has its own characteristic chiral signal that is measurable by CD and this difference might be due to heterogeneous exosomal protein expression on its surface.

This is confirmed by using SEM and chiral AuNPs with Annexin V capture exosomes on their outer edge (FIG. 4E). Thus, the CD peaks from the baseline (FIG. 4G) can be associated with successful capture of A549 derived exosomes on chip.

As the Annexin V-PS affinity is Ca²⁺ dependent, the isolated exosomes can be easily released following Ca²⁺ chelation by EDTA. Indeed, the devices can be restored to their original signal, indicating the ability to be reused. The capture-release cycle can be used to increase the accuracy of detection. This also implies that on-chip exosome binding only leads to change of CD signatures and analysis of this change can be used for label-free exosome detection and rapid profiling.

To quantitatively profile exosome binding in the same source of exosomes, signals of Δλ and ΔCD at several exosome numbers (10²-10⁸/device) are analyzed (FIG. 4H-4I). Compared to previous ELISA and similar optical sensing methods, the CDEXO platform-based sensing is more sensitive and requires smaller amounts of exosomes per sensing (10²-10⁴).

The exosome capturing performance of the microfluidic device is quantitatively analyzed using different exosome samples in terms of recovery rate. Recovery rate is defined as the fraction of release resultant concentration to sum of the capture effluent and release resultant concentrations from the microfluidic device. This describes how efficiently the present device is able to capture and release exosomes specifically. For evaluating these quantities, Nanoparticle Tracking Analysis (NTA) on Malvern's NanoSight is used and evaluated size distribution and exosomal concentration of samples. The average recovery rate for CDEXO chips is found to be around 70%, whereas the average recovery rate for control devices with no Annexin V conjugation is found to be around 10% (FIG. 4J).

### CDEXO Isolation and Profiling of Exosomes

Exosome capture is analyzed based on Δλ and ΔCD in a range of 500 nm-550 nm. After flowing exosomes through the devices, a sharp positive increase in CD peak as well as an increase in the spectral position of the peak maximum are observed (FIG. 3E). The majority of the analysis comes from the more notable peaks present in the range of 500 nm-700 nm. The two well-studied cancer-cell derived exosomes (A549 and H3255) exhibit a distinctly greater shift from the baseline than the healthy MRC5-derived exosomes, indicating that the difference between two types of cancer exosomes can be recognized (FIG. 4K). Interestingly, exon 19 deletion exosomes (H1650) exhibit a smaller shift from the baseline and its shift was similar to the healthy MRC5-derived exosomes. Additionally, it appears that those exosomes exhibiting EGFR point mutation are even more so distinguished than the wild type and exon 19 deletion, exhibiting a larger degree of peak shift. Note also, a negative CD peak present can also be analyzed in respect to the spectral changes in presence of exosomes. It is unclear why the H3255 having point mutation in EGFR could present this additional negative peak, but it provides a distinct marker that can be of use in distinguishing between cancers of differing severity.

Similarly, to the positive side of the bisignate CD spectrum, evaluation of negative CD peak in 500 nm-550 nm range reveals a significant increase in the magnitude of the peak after the device is spiked with certain cell-line derived exosome samples (FIG. 4L), the change in magnitudes of these valleys normalized against the original magnitudes is much lower with healthy cell line-derived exosome samples as compared to those of cancer cell line-derived exosome samples.

### Clinical Validation of the CDEXO Detection of Exosomes

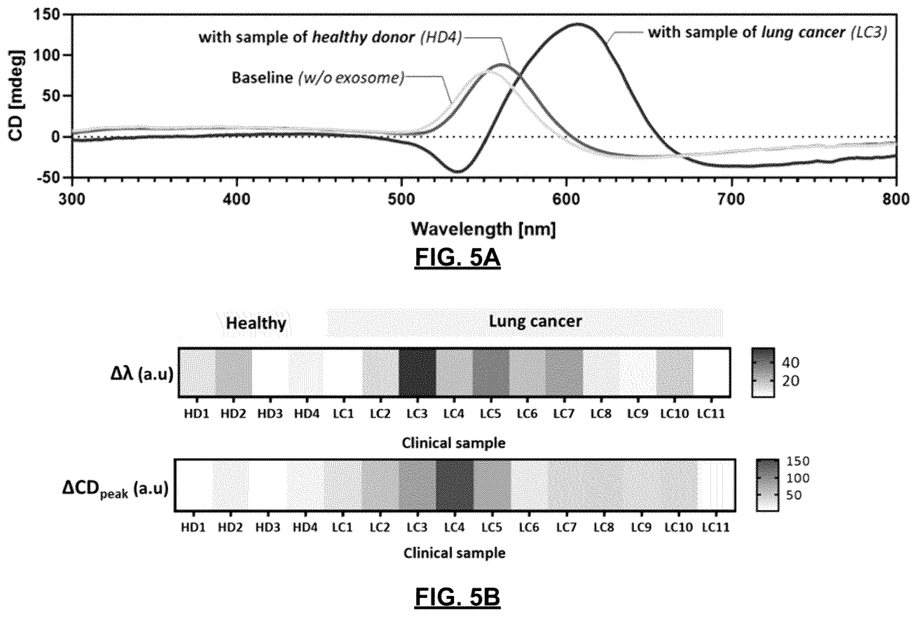

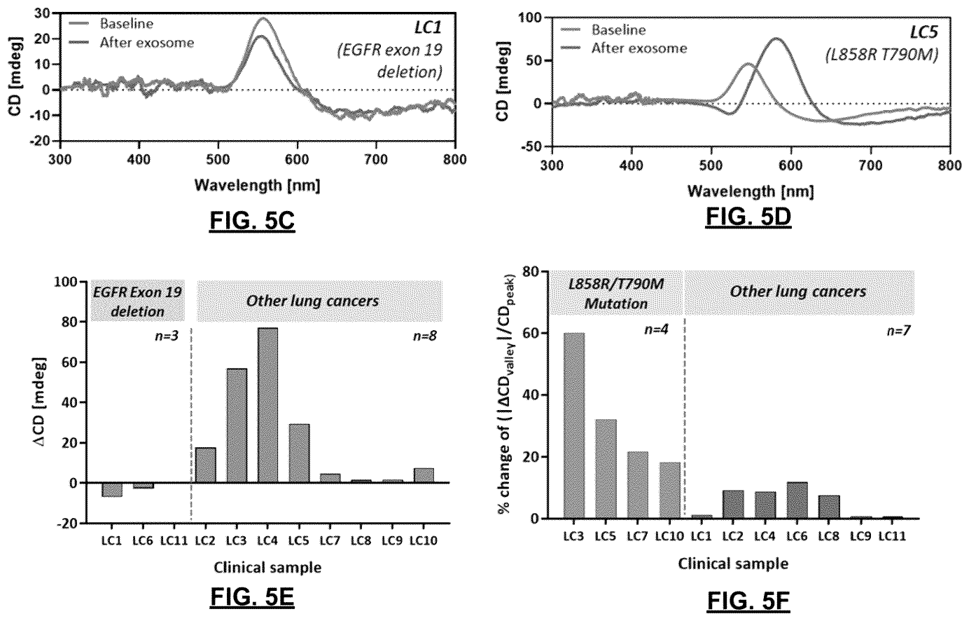

Trials incorporating clinical samples towards the present technology serve to verify the applicability of the microfluidic devices as a potential diagnostic tool (FIGS. 5A-5F). The representative CD peaks from a healthy donor (HD4) and a sample with lung cancer (LC3) are shown in FIG. 5A with a baseline signal. A noticeable positive increase in ΔCD as well as a positive A, is observed for the cancer cell secreted exosomes. FIG. 5B shows the relative comparisons to baseline values for each clinical sample in terms of shift of the peak and CDₚₑₐₖ magnitude change. Lung cancer samples show greater peak shifts compared to healthy donors (A, =24.57 vs. 10.00, P-value: 0.1468); however, there are some lung cancer patients showing minimal change in peak shift as compared to those of the healthy donors. Besides the shift of the peak signal, ΔCDₚₑₐₖ is also evaluated. Additionally, the CD magnitude changes are divided by the original magnitudes of each device and it demonstrates that the lung cancer samples overwhelmingly show larger % changes than the healthy donors, with only one lung cancer sample showing a comparable % change to that of one other healthy donor. However, in all cases, lung cancer samples showed higher % changes than that of healthy donors (% change of ΔCDₚₑₐₖ=58.36 vs. 7.693, P-value: 0.0617). Based on these results, the chiroplasmonic CDEXO chip can be used for cancer cell-derived exosome detection with % change of CDₚₑₐₖ magnitude being a more straightforward and reliable metric in distinguishing between healthy and cancerous samples than the current comparable methods that target certain cancer associated receptor or require complex computational analysis to classify one from another.

### Profiling of the Plasma Exosomes in Cancer Using CDEXO Chip

After noticing the differences between A549 and H3255 cell lines, further explorations are conducted in order to quantify the extent to which EGFR mutations may affect CD signatures of exosomes on chiral AuNPs. FIGS. 5C-5D depict the CD signal changes for specific subgroups in EGFR such as EGFR exon 19 deletion (FIG. 5C) and exon 21 substitutional point mutations in the EGFR gene (FIG. 5D), which account for 90% of all EGFR mutations. The ΔCDₚₑₐₖ and Δλ, vary between these three cases. Interestingly, the samples with EGFR mutation display the negative peak in 500 nm-550 nm range, which seems to be characteristic of the EGFR exon 19 deletion-mutated exosome samples. FIG. 5E depicts the ΔCD for exon 19 deletion in the EGFR gene. Unlike the other 8 lung cancer cases, samples with EGFR exon 19 deletion show minimal peak magnitude change from the baseline (−4.784 vs. 37.21, P-value: 0.1166). The percent change of a characteristic negative peak (valley) compared to its conventional positive peak (peak) is demonstrated. The sample percent change evaluation used for CDₚₑₐₖ analysis is now applied to the change in magnitude of the negative CD peaks (CD_{valley}) present on certain types of samples after processing with clinical samples. The samples having EGFR point mutations have significantly greater values in the % change than other cancer cases (37.98 vs. 7.768, P-value: 0.0290) and based on the results with cell line-derived exosomes (FIG. 4E), it is possible that this EGFR point mutations have the effect of inducing this large percent increase in negative peak magnitude. It is unclear as to exactly why these specific point mutations and deletion in both cases could cause such radical changes in the CD signals, but it can most likely be attributed to large structural changes in the EGFR proteins due to the improper folding that these mutations are likely to induce. This issue merits further exploration if this device is to be implemented further, as understanding how specific mutations and their induced structural changes can affect the CD signal are expected to increase the utility and specificity of the CDEXO platform.

A microfluidic device prepared in accordance with certain aspects of the present disclosure may include a layer of chiral AuNPs, which affords sensitive and accurate detection of lung cancer-associated exosomes from plasma samples. The cancer specificity enabled by the Annexin V conjugation allows for anchoring lung cancer associated exosomes on the sensing regions of the device. The resulting strong and characteristic CD peaks, which arise from specific interactions between exosomal surface proteins and chiral AuNPs, facilitate sensitive and in-depth profiling of target exosomes, including EGFR mutation expression. Given that cancer exosomes and their innate molecular information may play an important role in cancer progression, it provides new ways for screening and diagnosing disease status enabling liquid biopsy. CDEXO chips afford straightforward exosome profiling that can be readily extended to other cancers. Besides clinical significance, the same methodology can also be applied to further exploration of exosomes roles in various diseases.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A microfluidic device comprising:
a microfluidic channel comprising at least one surface having a plurality of chiral nanoparticles disposed thereon, wherein the plurality of chiral nanoparticles each comprise a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof, and a targeting ligand associated with the plurality of chiral nanoparticles that is capable of binding to a bioactive target analyte in a biological fluid sample, wherein the bioactive target analyte indicates a presence of cancerous cells or mutated proteins in the biological fluid sample.

2. The microfluidic device of claim 1, wherein the microfluidic channel comprises a multilayered coating formed by a layer-by-layer deposition process that comprises a plurality of positive layers interspersed with a plurality of negative layers, wherein an exposed surface defined by the multilayered coating comprises the plurality of chiral nanoparticles having a positive charge.

3. The microfluidic device of claim 2, wherein at least one layer of the plurality of positive layers comprises a cationic poly(dimethyldiallylammonium chloride) (PDDA) and at least one layer of the plurality of negative layers comprises an anionic polystyrene sulfonate (PSS) and the at least one surface of the microfluidic channel is plasma etched.

4. The microfluidic device of claim 1, wherein the microfluidic channel is formed on a microchip.

5. The microfluidic device of claim 1, wherein the bioactive target analyte is selected from the group consisting of: phosphatidylserine (PS), tetraspanin proteins, epithelial cancer adhesion molecule (EpCAM), epidermal growth factor receptor (EGFR), and combinations thereof.

6. The microfluidic device of claim 1, wherein the plurality of chiral nanoparticles comprise chiral gold nanoparticles functionalized with mercaptoundecanoic acid (MUA) reacted with 1-ethyl-3-(3-dimethylaminopropyl)carbodiimide (EDC) and N-hydroxysuccinimide (NHS).

7. The microfluidic device of claim 1, wherein the targeting ligand is selected from the group consisting of: Annexin V, anti-CD63, anti-CD81, anti-CD9, anti-CD56, anti-CD-133, anti-EpCAM, anti-EGFR, anti-vimentin, and combinations thereof.

8. The microfluidic device of claim 1, wherein the bioactive target analyte comprises phosphatidylserine (PS) and the targeting ligand comprises Annexin V.

9. The microfluidic device of claim 8, wherein the targeting ligand further comprises deglycosylated avidin associated with biotin that is associated with Annexin V.

10. A method of detecting a target bioactive analyte in a biological fluid sample obtained from a subject, the method comprising:
passing a biological fluid sample through a microfluidic channel comprising at least one surface having a plurality of chiral nanoparticles disposed thereon and directing circularly polarized light at the microfluidic channel while the biological fluid sample is disposed therein to measure a first level of at least one of magnitude of circular dichroism or peak wavelength, wherein the plurality of chiral nanoparticles each comprises a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof, and a targeting ligand that is capable of binding to a bioactive target analyte optionally present in the biological fluid sample; and
comparing the first level of at least one of magnitude of circular dichroism or peak wavelength to a baseline level of at least one of magnitude of circular dichroism or peak wavelength in the microfluidic channel in the absence of the biological fluid sample, wherein a difference between the first level and the baseline level indicates a presence of the bioactive target analyte that indicates a presence of cancerous cells or mutated proteins in the biological fluid sample.

11. The method of claim 10, further comprising measuring the baseline level of at least one of magnitude of circular dichroism or peak wavelength by directing circularly polarized light at the microfluidic channel in the absence of the biological fluid sample.

12. The method of claim 10, wherein the first level is a peak wavelength measured in a range of greater than or equal to about 520 nm to less than or equal to about 1.4 micrometers.

13. The method of claim 10, wherein the first level is a peak magnitude of circular dichroism and the baseline level is a peak magnitude of circular dichroism and the comparing shows a difference in peak magnitudes of circular dichroism between the first level and the baseline level.

14. A method for forming a microfluidic device for detecting a bioactive target analyte in a biological fluid sample obtained from a subject, the method comprising:
applying a first charged material having a first polarity to at least one surface of a microfluidic channel on a substrate having a second polarity opposite to the first polarity;
applying a second charged material having the second polarity over the first charged material in a layer-by-layer process on the at least one surface, wherein the first charged material and the second charged material are distinct from one another and define a layered coating;
applying a plurality of chiral nanoparticles over the layered coating, so that the plurality of chiral nanoparticles are exposed to the microfluidic channel and each comprises a light-absorbing material selected from the group consisting of: gold, silver, copper, nickel, iron, carbon, platinum, silicon, CdTe, CdSe, CdS, HgTe, HgSe, HgS, PbTe, PbSe, PbS, MoS₂, FeS₂, FeS, FeSe, and combinations thereof; and
functionalizing the plurality of chiral nanoparticles and associating each nanoparticle of the plurality with a targeting ligand that is capable of binding to the bioactive target analyte in the biological fluid sample, wherein the bioactive target analyte indicates a presence of cancerous cells or mutated proteins in the biological fluid sample.

15. The method of claim 14, wherein the first charged material comprises a cationic poly(dimethyldiallylammonium chloride) (PDDA) and the second charged material comprises an anionic polystyrene sulfonate (PSS) and the plurality of chiral nanoparticles are cationic and have the first polarity.

16. The method of claim 14, wherein the plurality of chiral nanoparticles are stabilized with a shape-directing ligand selected from the group consisting of: L-cysteine, D-cysteine, L-penicillamine, D-penicillamine, dopamine, L-carnosine, amyloid peptide monomers, amino-acid derivatives, and combinations thereof.

17. The method of claim 14, further comprising plasma etching the at least one surface of the microfluidic channel.

18. The method of claim 14, wherein the substrate is a microchip and the microfluidic channel is formed on the microchip.

19. The method of claim 14, wherein the plurality of chiral nanoparticles comprise chiral gold nanoparticles and the functionalizing comprises reacting the chiral gold nanoparticles with mercaptoundecanoic acid (MUA) followed by reacting with 1-ethyl-3-(3-dimethylaminopropyl)carbodiimide (EDC) and then reacting with N-hydroxysuccinimide (NHS) to form a plurality of functionalized chiral gold nanoparticles.

20. The method of claim 14, wherein the targeting ligand is selected from the group consisting of: Annexin V, anti-CD63, anti-CD81, anti-CD9, anti-CD56, anti-CD-133, anti-EpCAM, anti-EGFR, anti-vimentin, and combinations thereof.

21. The method of claim 14, wherein the bioactive target analyte comprises phosphatidylserine (PS) and the targeting ligand comprises Annexin V reacted with the plurality of functionalized chiral gold nanoparticles.

22. The method of claim 21, wherein the targeting ligand further comprises first associating deglycosylated avidin with the plurality of functionalized chiral gold nanoparticles, then associating biotin with the deglycosylated avidin, followed by associating the biotin with Annexin V capable of binding to the bioactive target analyte.

23. The method of claim 14, further comprising forming the plurality of chiral nanoparticles by growing triangular nanoplate precursors by adding gold precursor, a reductant, and a shape-directing ligand, followed by encapsulating the plurality of chiral nanoparticles in positively charged bilayer micelles.
